(19) United States
(12) Patent Application Publication
(10) Pub. No.: US 2014/0220680 A1
(43) Pub. Date: Aug. 7, 2014

(54) XENO-FREE AND A FEEDER FREE SELF-RENEWAL EXTRACELLULAR MATRIX FOR LONG-TERM MAINTENANCE OF UNDIFFERENTIATED HUMAN PLURIPOTENT STEM CELLS AND METHOD OF SYNTHESIZING THE SAME

(71) Applicants: HOSSEIN BAHARVAND, TEHRAN (IR); MOHAMMAD PAKZAD, TEHRAN (IR)

(72) Inventors: HOSSEIN BAHARVAND, TEHRAN (IR); MOHAMMAD PAKZAD, TEHRAN (IR)

(73) Assignee: ROYAN RESEARCH, TEHRAN (IR)

(21) Appl. No.: 13/757,973

(22) Filed: Feb. 4, 2013

(51) Int. Cl. C12N 5/0735

(52) U.S. Cl. CPC C12N 5/0606
USPC 435/366

(57) ABSTRACT

The embodiments herein provide a xeno-free and a feeder free self-renewal extracellular matrix for long-term maintenance of undifferentiated Human Embryonic Stem Cells (hESCs) and undifferentiated Human Pluripotent Stem Cells (hPSCs) and a method of synthesizing the same. The extracellular matrix includes a conditioned medium comprising a neurobasal medium, a DMEM/F12 medium, a plurality of additives and a plurality of feeder cells. The plurality of additives are L-glutamine, β-mercaptoethanol, nonessential amino acids and insulin-transferrin-selenite. The feeder cells are Human Dermal Fibroblasts (HDFs). The conditioned medium may be added with a Rho-associated Coiled Kinase (ROCK) inhibitor Y-27632. The extracellular matrix is in the form of a gel. The method comprises preparing a conditioned medium and incubating the conditioned medium for 24 hours or 72 hours at 37° C. The method comprises coating a plate with the prepared conditioned medium for 5 min or 15 min.

### CROSS-REFERENCE TO RELATED APPLICATION

This application claims the priority under 35 USC 119(e) of U.S. Provisional Application Ser. No. 61/676,900 filed Jul. 28, 2012, which included by reference herein.

### BACKGROUND

1. Technical Field

The embodiments herein generally relate to a field of extracellular matrix for preserving biological cells. The embodiments herein particularly relate to an extracellular matrix for long term maintenance of the undifferentiated stem cells. The embodiments herein more particularly relate to a xeno-free and a feeder free self-renewal extracellular matrix for a long-term maintenance of the undifferentiated Human Embryonic Stem Cells (hESCs) and Human Pluripotent Stem Cells (hPSCs). The embodiments herein also relate to a method of synthesizing the xeno-free and a feeder free self-renewal extracellular matrix for a long-term maintenance of the undifferentiated Human Embryonic Stem Cells (hESCs) and Human Pluripotent Stem Cells (hPSCs).

2. Description of the Related Art

Human Embryonic Stem Cells (hESCs) and Human Induced Pluripotent Stem Cells (hiPSCs) have provided fascinating possibilities and tools to study the human development and genetic diseases. The use of Human Embryonic Stem Cells (hESCs) and Human Induced Pluripotent Stem Cells (hiPSCs) have also provided opportunities for the development of toxicological and pharmaceutical applications, as well as in developing an in vitro disease modeling. There has been tremendous interest in developing the culture systems for growing, culturing, maintaining and preserving the stem cells. The first report on the generation of the human embryonic stem cells (hESCs) from a pre-implantation of embryos and human induced pluripotent stem cells (hiPSCs) from a reprogramming of the somatic cells to decrease the cell heterogeneity and producing the quality cells at a scale that is suitable for the biomedical applications was provided. Since then, the researchers have been engaged/involved in developing a culture medium that can retain the undifferentiated stem cells for a long period of time. The various culture conditions were developed for self-renewal and maintenance of pluripotency such as use of mouse feeder cells layers or human feeder cell layers to feeder and serum-free conditions.

A progress in developing the defined conditions for hESC expansion led to the production of several culture media such as X-VIVO 10, mTeSR, and STEMPRO. But it remained a challenge to identify an optimum substrata to propagate the hPSCs in the feeder-free conditions. For this purpose, a typical extracellular matrix (ECM) in use is a MatriGel (MG). The MatriGel is a complex mouse sarcoma cell basement membrane extract comprising the various ECM proteins and growth factors. The MatriGel is the sole gold standard ECM for a long-term feeder-free expansion of the undifferentiated hPSCs. The various research groups used various alternatives to replace the MatriGel. The alternatives such as laminin, fibronectin, vitronectin, animal-derived matrices, ECM derived from mouse embryonic fibroblasts and human foreskin fibroblast (HFF), a mixture of human collagen IV, vitronectin, fibronectin, laminin and human serum coating have been used to replace the use of feeder cells in the hESC cultures. A feeder-free derivation of hESC lines is also successfully described using a mouse-derived matrix and a chemically defined xeno-free culture medium and a combination of human laminin, collagen IV, fibronectin and vitronectin matrix.

But these biological materials have limited scalability or are not available in every lab. These biological materials have a high batch-to-batch variability and are very costly for a routine culture. Moreover the animal-derived materials expose the hPSCs to potentially hazardous pathogens that allows the transfer of immunogenic epitopes. Most of these ECMs are generally effective for only short-term propagation. The researchers are still searching for the xeno-free culture vessel coatings that do not induce a cellular differentiation in hPSCs and save a chromosomal integrity.

Recently several groups have reported that a feeder-free propagation of hESCs is achievable with a recombinant human laminin 511. The recombinant human laminin 511 is a defined glycosaminoglycan-binding substratum (GKKQRFRHRNRKG) peptide that is derived from the RGD (Rat Genome Database) containing the adhesion domains of bone sialoprotein and vitronectin which are covalently linked to an acrylate surface. The monomers with a high acrylate content have a moderate wettability and employ an integrin α5β3 and α5β5 engagement with an adsorbed vitronectin, a synthetic polymer substrate named poly[2-(methacryloyloxy)ethyl dimethyl-(3-sulfopropyl)ammonium hydroxide] (PMEDSAH) and UV/ozone radiation modification of typical cell culture plastics. However, these materials also have most of the aforementioned problems for a long-term routine expansion of hPSCs. Therefore the attempts to find the substrates suitable for a simple promotion of a long-term hESC and hiPSC self-renewal have attracted a great interest.

Hence there is a need to develop an extra cellular matrix that provides a long term expansion of the undifferentiated hESCs and hiPSCs. There is also a need to provide an extracellular matrix that is simple to synthesize and readily available in the market.

The above mentioned shortcomings, disadvantages and problems are addressed herein and which will be understood by reading and studying the following specification.

### OBJECTIVES OF THE EMBODIMENTS

The primary object of the embodiments herein is to provide a xeno-free and a feeder free self-renewal extracellular matrix for long-term maintenance of the undifferentiated Human Embryonic Stem Cells (hESCs) and undifferentiated Human Pluripotent Stem Cells (hPSCs).

Another object of the embodiments herein is to provide a simple, robust, repeatable and cost-effective extracellular matrix for the maintenance of the Human Pluripotent Stem Cells.

Yet another object of the embodiments herein is to provide an extracellular matrix comprising a basal conditioned medium and the human feeder cells.

Yet another object of the embodiments herein is to provide an extracellular matrix that is simple to synthesize, easily available and does not induce a cellular differentiation in the hPSCs and save a chromosomal integrity.

Yet another object of the embodiments herein is to provide an extracellular matrix for culturing the hPSCs that can be cryopreserved and successfully thawed back onto the matrix for a further expansion or differentiation which is important for a production of a clinical grade hPSC banks.

Yet another object of the embodiments herein is to provide an extracellular matrix which is prepared from a neurobasal medium without using any serum or KockOut Serum Replacement (KOSR).

Yet another object of the embodiments herein is to provide an extracellular matrix coated plates to maintain the self-renewal of hPSCs for a period of one year when preserved at a room temperature.

Yet another object of the embodiments herein is to provide a method of synthesizing an extracellular matrix for long term preserving and maintaining the undifferentiated hPSCs.

These and other objects and advantages of the embodiments herein will become readily apparent from the following detailed description taken in conjunction with the accompanying drawings.

### SUMMARY

The various embodiments herein provide a xeno-free and a feeder free self-renewal extracellular matrix for long-term maintenance of undifferentiated Human Embryonic Stem Cells (hESCs) and undifferentiated Human Pluripotent Stem Cells (hPSCs). The embodiments herein also provide a method of synthesizing a composition for a xeno-free and a feeder free self-renewal extracellular matrix for long-term maintenance of undifferentiated Human Embryonic Stem Cells (hESCs) and Human Pluripotent Stem Cells (hPSCs). Moreover, the embodiments herein provide a method of preparing culture plates for culturing and long-term preserving and maintaining the human stem cells.

According to one embodiment herein, a xeno-free and a feeder free self-renewal extracellular matrix for long-term maintenance of the undifferentiated Human Embryonic Stem Cells (hESCs) and undifferentiated Human Pluripotent Stem Cells (hPSCs) comprises a conditioned medium. The conditioned medium comprises a basal medium, a nutrition providing medium, a plurality of additives and a plurality of feeder cells. The basal medium is neurobasal medium. The nutrition providing medium is Dulbecco's Modified Eagle Medium/F12 (DMEM/F12). The plurality of feeder cells are Human Dermal Fibroblasts (HDFs). The plurality of feeder cells are inactivated by mitomycin C for 2 hours. The plurality of additives are L-glutamine, β-mercaptoethanol, nonessential amino acids and insulin-transferrin-selenite. The nonessential amino acids (NEAAs) are alanine, asparagines, aspartic acid, cysteine, cyctine, glutamine, glutathione, glycine, histidine, proline, serine, taurine and threonine. The conditioned medium is added with a Rho-associated Coiled Kinase (ROCK) inhibitor Y-27632. The conditioned medium is derived from human feeder cells. The human feeder cells are Human Dermal Fibroblasts (HDFs). The Human Dermal Fibroblasts are taken in an amount selected from the group consisting of a high density and a low density. The high density is 50000 cell/cm² and the low density is 5000 cell/cm². The conditioned medium maintains undifferentiated Human Embryonic Stem Cells (hESCs) and an Human Pluripotent Stem Cells (hPSCs) for a period of one year at room temperature. The extracellular matrix is in the form of a gel.

According to an embodiment herein, a method of synthesizing a xeno-free and a feeder free self-renewal extracellular matrix for long-term maintenance of undifferentiated Human Embryonic Stem Cells (hESCs) and Human Pluripotent Stem Cells (hPSCs) comprises preparing a conditioned medium. The conditioned medium is prepared by adding a basal medium, a nutrition providing medium, a plurality of additives and a plurality of feeder cells together. The basal medium is neurobasal medium. The nutrition providing medium is Dulbecco's Modified Eagle Medium/F12 (DMEM/F12). The plurality of feeder cells are Human Dermal Fibroblasts (HDFs) which are inactivated by mitomycin C for 2 hours. The conditioned medium is incubated for a pre-determined amount of time at a pre-determined temperature. The pre-determined amount of time is 24 hours and 72 hours. The pre-determined amount of temperature is 37° C. The plurality of additives includes L-glutamine (L-Gln), β-mercaptoethanol (β-ME), nonessential amino acids (NEAAs), and insulin-transferrin-selenite (ITS). The nonessential amino acids (NEAAs) are alanine, asparagines, aspartic acid, cysteine, cyctine, glutamine, glutathione, glycine, histidine, proline, serine, taurine and threonine. The extracellular matrix maintains an undifferentiated Human Embryonic Stem Cells (hESCs) and a Human Pluripotent Stem Cells (hPSCs) for a period of one year at room temperature. The extracellular matrix is in the form of a gel.

According to an embodiments herein, a method of synthesizing a xeno-free and a feeder free self-renewal extracellular matrix for long-term maintenance of undifferentiated Human Embryonic Stem Cells (hESCs) and Human Pluripotent Stem Cells (hPSCs) further comprises preparing a culture plate for culturing an Human Embryonic Stem Cells (hESCs) and a Human Pluripotent Stem Cells (hPSCs). The step of preparing the culture plates comprises coating a plate with a conditioned medium for a predetermined amount of time. The coating is a simple coating. The coating is layer-by-layer coating. The predetermined amount of time is 5 min and 15 min.

According to one embodiment herein, a method of providing a simple coating on a culture plate comprises adding the conditioned medium on a plate. The plate is swirled to evenly spread the conditioned medium. The plate is incubated at a predetermined temperature for the predetermined amount of time. The pre-determined temperature is 37° C. The predetermined amount of time is 5 min and 150 min. The plate is dried. The plate is dried at room temperature for 15-20 min.

According to another embodiment herein, a method of providing a layer-by-layer coating on a culture plate comprises adding the conditioned medium on a plate. The plate is swirled to evenly spread the conditioned medium. The plate is incubated at a predetermined temperature and for the predetermined amount of time. The pre-determined temperature is 37° C. The predetermined amount of time is 5 min and 150 min. The plate is dried. The plate is dried at room temperature for 15-20 min. The plate is recoated after drying for a plurality of time. The conditioned medium is further added with a Rho-associated Coiled Kinase (ROCK) inhibitor Y-27632. The plates are coated with >10 KD and >30 KD fractionated conditioned medium.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart indicating the various steps in a method of synthesizing a xeno-free and a feeder free self-renewal extracellular matrix for long-term maintenance of undifferentiated Human Embryonic Stem Cells (hESCs) and Human Pluripotent Stem Cells (hPSCs), according to an embodiment herein.

FIG. 2 illustrates a histogram indicating the Normalized plating efficiency (PE) of the neurobasal medium and DMEM/F12 medium on the attachment of hPSCs, according to the embodiments herein.

FIG. 3A illustrates an image indicating the feeder cells derived from Human Dermal Fibroblasts (HDFs) at a low density, according to the embodiments herein.

FIG. 3B illustrates an image indicating the feeder cells derived from Human Dermal Fibroblasts (HDFs) at a high density, according to the embodiments herein.

FIG. 3C illustrates an image indicating the feeder cells derived from Human Foreskin Fibroblasts (HFFs) at a low density, according to the embodiments herein.

FIG. 3D illustrates an image indicating the feeder cells derived from Human Foreskin Fibroblasts (HFFs) at a high density, according to the embodiments herein.

FIG. 4A illustrates a histogram indicating the plating efficiency of hPSCs plated on conditioned medium under different conditions such as cell density, time of conditioning and coating time, according to the embodiments herein.

FIG. 4B illustrates an image of a dish indicating a growth of hPSCs stained with Alkaline phosphatase stain in the conditioned medium (CM) with high cell density and conditioned for 72 h, according to the embodiments herein.

FIG. 4C illustrates an image of a dish indicating a growth of hPSCs stained with Alkaline phosphatase stain in the conditioned medium with high cell density conditioned for 24 h, according to the embodiments herein.

FIG. 4D illustrates a histogram indicating the effect of dilution of the CM in presence and absence of ROCKi with a presence and absence of Y-276320 in coating the CM and the effect of different coating method, according to the embodiments herein.

FIG. 4E illustrates a histogram indicating the percentage of Ki-67 positive cells for the Rogel and the matrigel, according to the embodiments herein.

FIG. 4F illustrates a histogram indicating the cloning efficiency of the R1-hiPSC4 and Royan H6 grown in the CM and MG, according to the embodiments herein.

FIG. 4G illustrates a histogram indicating the plating efficiency of the grown hESC and hiPSC cell lines on the CM and the MG, according to the embodiments herein.

FIG. 5A-FIG. 5D illustrates are the images indicating the growth morphology of the hPSC colonies post-subculturing, where FIG. 5A denotes grade-A, FIG. 5B denotes grade-B, FIG. 5C denotes grade-C and FIG. 5D denotes grade-D, according to the embodiments herein.

FIG. 5E illustrates a histogram indicating the percentages of the undifferentiated and differentiated colonies in presence of HFF Rogel or conditioned medium derived from HFF as feeder cells and HDF Rogel or the conditioned medium derived from HDF as feeder cells and MG, according to the embodiments herein.

FIG. 5F illustrates a histogram indicating the Plating Efficiency of the cultured medium grown in presence of HFF Rogel or conditioned medium derived from HFF as feeder cells and HDF Rogel or the conditioned medium derived from HDF as feeder cells and MG, according to the embodiments herein.

FIG. 5G illustrates a histogram indicating the percentages of the undifferentiated and differentiated colonies in presence of HFF Rogel or conditioned medium derived from HFF as feeder cells and HDF Rogel or the conditioned medium derived from HDF as feeder cells at passage number 10 and passage number 20 and MG, according to the embodiments herein.

FIG. 5H illustrates a histogram indicating the Plating Efficiency of the cultured medium grown in presence of HFF Rogel or conditioned medium derived from HFF as feeder cells and HDF Rogel or the conditioned medium derived from HDF as feeder cells at passage number 10 and passage number 20 and MG, according to the embodiments herein.

FIG. 5I illustrates a histogram indicating the effect of age of feeder cells on CM after the preparation of inactivated HDF by mitomycin C, according to the embodiments herein.

FIG. 6 illustrates a histogram indicating the influence of temperature and time of the storage on the quality of CM, according to the embodiments herein.

FIG. 7A and FIG. 7B illustrates the images of the colonies on CM coated dishes indicating the effect of the enzymes treatment on the colonies where FIG. 7A illustrates the image of colony before the enzymatic treatment and FIG. 7B illustrates an image of the colony after the enzymatic treatment, according to the embodiments herein.

FIG. 7C illustrates an image of the colony cut by STEMPRO® EZPassage™ tool, according to an embodiment herein.

FIG. 7D illustrates an image of the colony pieces ready to transfer to the new vessels, according to an embodiment herein.

FIG. 7E illustrates an image of a part of the colony after two days of culturing on RoGel or CM, according to an embodiment herein.

FIG. 7F illustrates an image of a part of the colony after seven days of culturing on RoGel or CM, according to an embodiment herein.

FIG. 8A-FIG. 8D illustrates the images of the grown colonies in presence of CM or the RoGel with other commercially available and chemically defined mediums, where FIG. 8A illustrates an image indicating the growth of a colony when cultured on CM containing DMEM/F12 in addition with KOSR, where FIG. 8B illustrates an image indicating the growth of a colony when cultured on CM in presence of mTeSR1, where FIG. 8C illustrates an image indicating the growth of a colony on MG in presence of DMEM/F 12 and KOSR and where FIG. 8D illustrates an image indicating the growth of a colony on MG in presence of mTeSR1, according to embodiments herein.

FIG. 8E illustrates a histogram indicating the effect of compatibility of CM with DMEM/F12 and mTeSR culture mediums, according to the embodiments herein.

FIG. 8F illustrates a histogram indicating the doubling time population in presence of CM with DMEM/F12 and mTeSR culture mediums, according to the embodiments herein.

FIG. 8G illustrates a histogram indicating the expression of sternness-specific marker i.e. NANOG, according to the embodiments herein.

FIG. 9A illustrates a photograph indicating the phase contrast of hESCs i.e. Royan H6 grown on the CM, according to an embodiment herein.

FIG. 9B illustrates a photograph indicating the phase contrast of hESCs i.e. Royan H6 grown on MG, according to the embodiment herein.

FIG. 9C illustrates a photograph indicating the hESCs or Royan H6 cell line by Alkaline Phosphatase (ALP) staining on the CM, according to an embodiment herein.

FIG. 9D illustrates a photograph indicating the hESCs or Royan H6 cell line by Alkaline Phosphatase (ALP) staining on the CM at a larger scale, according to an embodiment herein.

FIG. 9E illustrates a photograph indicating the hESCs or Royan H6 cell line by Alkaline Phosphatase (ALP) staining on the MG, according to an embodiment herein.

FIG. 9F illustrates a photograph indicating the hESCs or Royan H6 cell line by Alkaline Phosphatase (ALP) staining on the MG at a larger scale, according to an embodiment herein.

FIG. 9G illustrates a histogram indicating an evaluation of the cloning efficiency for cultured hPSCs, according to an embodiment herein.

FIG. 10A illustrates an image indicating the phase contrast of hESCs, according to an embodiment herein.

FIG. 10B illustrates an image indicating the phase contrast of hESCs at a magnified scale, according to an embodiment herein.

FIG. 10C illustrates an image indicating the expression of hESCs in presence of OCT4 marker, according to the embodiments herein.

FIG. 10D illustrates an image indicating the stained nuclei with DAPI, according to the embodiments herein.

FIG. 10E illustrates an image indicating the expression of hESCs in presence of OCT4 and DPAI, according to the embodiments herein.

FIG. 10F illustrates an image indicating the expression of hESCs in presence of SSEA3 marker, according to the embodiments herein.

FIG. 10G illustrates an image indicating the stained nuclei with DAPI, according to the embodiments herein.

FIG. 10H illustrates an image indicating the expression of hESCs in presence of SSEA3 marker and DPAI stain, according to the embodiments herein.

FIG. 10I illustrates an image indicating the alkaline phosphatase staining of the hSECs, according to the embodiments herein.

FIG. 10J illustrates an image indicating the expression of cells in presence of TRA-1-81 marker, according to the embodiments herein.

FIG. 10K illustrates an image indicating the stained nuclei with DAPI stain, according to the embodiments herein.

FIG. 10L illustrates an image indicating the expression of hESCs in presence of TRA-1-81 marker and DPAI stain, according to the embodiments herein.

FIG. 11A illustrates a karyotype of Royan H5 cell line, according to the embodiments herein.

FIG. 11B illustrates a karyotype of Royan H6 cell line, according to the embodiments herein.

FIG. 11C illustrates a karyotype of R1-hiPSC1 cell line, according to the embodiments herein.

FIG. 11D illustrates a karyotype of R1-hiPSC4 cell line, according to the embodiments herein.

FIG. 11E illustrates a RT-PCR analysis of in vitro differentiation of hESCs i.e. Royan H5 cell lines by EB formation indicating the expression of the markers of the three embryonic germ layers, according to the embodiments herein.

FIG. 11F-FIG. 11I illustrates the images indicating the formation of teratomas and further indicating the presence of cells from all the three germ layers, where FIG. 11F illustrates a macroscopic view of the teratoma with distinct retinal pigment epithelium (RPE) on the teratoma, where FIG. 11G illustrates an image indicating the microscopic view of the formed teratoma showing the Retinal Pigment Epithelium (RPE), where FIG. 11H illustrates an image indicating the microscopic view of the formed teratoma showing the cartilage, where FIG. 11I illustrates an image indicating the microscopic view of the formed teratoma showing the intestinal epithelium, according to the embodiments herein.

FIG. 12A and FIG. 12B illustrates a diagrammatic representation of a Millipore's Amicon Ultra-15 centrifugal filter device indicating the fractionation of the CM, where FIG. 12A illustrates a diagrammatic representation of a Millipore's Amicon Ultra-15 centrifugal filter device indicating the fractionation of the CM to >10 KD and <10 KD based on the Molecular Weight of CM's proteins (MWCO) and where FIG. 12B illustrates a diagrammatic representation of a Millipore's Amicon Ultra-15 centrifugal filter device indicating the fractionation of the CM to >30 KD and <30 KD based on the Molecular Weight of CM's proteins (MWCO), according to an embodiment herein.

FIG. 12C-FIG. 12F illustrates the images of the colonies of the hPSCs grown on the fractionated CM, where FIG. 12C illustrates an image indicating the growth of hPSCs on the >10 KD fractionated CM, where FIG. 12D illustrates an image indicating the growth of hPSCs on the <10 KD fractionated CM, where FIG. 12E illustrates an image indicating the growth of hPSCs on the >30 KD fractionated CM and where FIG. 12F illustrates an image indicating the growth of hPSCs on the <30 KD fractionated CM, according to the embodiments herein.

FIG. 13A illustrates a phase contrast microscopy image of plate coated with simple layer of MG, according to the embodiments herein.

FIG. 13B illustrates a phase contrast microscopy image of plate coated with simple layer of Fresh CM, according to the embodiments herein.

FIG. 13C illustrates a phase contrast microscopy image of plate coated with >10 KD fractionated CM, according to the embodiments herein.

FIG. 13D illustrates a phase contrast microscopy image of plate coated with simple layer of CM, according to the embodiments herein.

FIG. 13E illustrates a phase contrast microscopy image of plate coated with simple layer of Fresh CM, according to the embodiments herein.

FIG. 13F illustrates a phase contrast microscopy image of plate coated with <10 KD fractionated CM, according to the embodiments herein.

FIG. 13G illustrates a phase contrast microscopy image of plate coated with CM using layer-by-layer method, according to the embodiments herein.

FIG. 13H illustrates a phase contrast microscopy image of plate coated with MG using layer-by-layer method, according to the embodiments herein.

FIG. 13I illustrates a phase contrast microscopy image of plate coated with >30 KD fractionated CM, according to the embodiments herein.

FIG. 13J illustrates a phase contrast microscopy image of plate coated with CM, according to the embodiments herein.

FIG. 13K illustrates a phase contrast microscopy image of plate coated with evaporated MG, according to the embodiments herein.

FIG. 13L illustrates a phase contrast microscopy image of plate coated with <30 KD fractionated CM, according to the embodiments herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The following detailed description uses certain abbreviations. The following is a list of full names of the abbreviations used to describe the various embodiments herein:
CM: Conditioned Medium
ECM: ExtraCellular Matrix
MG: MatriGel
hPSC: Human Pluripotent Stein Cell
hESC: Human Embryonic Stein Cell
hiPSC: Human induced Pluripotent Stein Cell
DMEM: Dulbecco's Modified Eagle Medium
KOSR: KockOut Serum Replacement
L-Gln: L-glutamine
β-ME: β-mercaptoethanol
NEAA: Nonessential Amino Acid
ITS: Insulin-Transferrin-Selenite
bFGF: basic Fibroblast Growth Factor
FITC: Fluorescein Isothiocyanate
IgM: Immunoglobulin M
IgG: Immunoglobulin G
Oct-4: Octamer-binding Transcription factor 4
SSEA-3: Stage-Specific Embryonic Antigens
DAPI: 4′,6-diamidino-2-phenylindole
EB: Embryoid Body
HFF: Human Foreskin Fibroblast

With respect to the following detailed description, the terms “Conditioned medium” and “Rogel” are used interchangeably.

The various embodiments herein provide a xeno-free and a feeder free self-renewal extracellular matrix for long-term maintenance of undifferentiated Human Embryonic Stem Cells (hESCs) and undifferentiated Human Pluripotent Stem Cells (hPSCs). The embodiments herein also provide a method of synthesizing a composition for a xeno-free and a feeder free self-renewal extracellular matrix for long-term maintenance of undifferentiated Human Embryonic Stem Cells (hESCs) and Human Pluripotent Stem Cells (hPSCs). Moreover, the embodiments herein provide a method of preparing culture plates for culturing and long-term preserving and maintaining the human stem cells.

According to one embodiment herein, a xeno-free and a feeder free self-renewal extracellular matrix for long-term maintenance of undifferentiated Human Embryonic Stem Cells (hESCs) and undifferentiated Human Pluripotent Stem Cells (hPSCs) comprises a conditioned medium. The conditioned medium comprises a basal medium, a nutrition providing medium, a plurality of additives and a plurality of feeder cells. The basal medium is neurobasal medium. The nutrition providing medium is Dulbecco's Modified Eagle Medium/F12 (DMEM/F12). The plurality of feeder cells are mitomycin C inactivated Human Dermal Fibroblasts (HDFs). The plurality of feeder cells are inactivated by mitomycin C for 2 hours. The plurality of additives are L-glutamine, β-mercaptoethanol, nonessential amino acids and insulin-transferrin-selenite. The nonessential amino acids (NEAAs) are alanine, asparagines, aspartic acid, cysteine, cyctine, glutamine, glutathione, glycine, histidine, proline, serine, taurine and threonine. The conditioned medium may further comprise with a Rho-associated Coiled Kinase (ROCK) inhibitor Y-27632. The conditioned medium is derived from human feeder cells. The human feeder cells are Human Dermal Fibroblasts (HDFs). The Human Dermal Fibroblasts are taken in an amount selected from the group consisting of a high density and a low density. The high density is 50000 cell/cm² and the low density is 5000 cell/cm². The conditioned medium maintains undifferentiated Human Embryonic Stem Cells (hESCs) and Human Pluripotent Stem Cells (hPSCs) for a period of one year at room temperature. The extracellular matrix is in the form of a gel.

According to an embodiment herein, a method of synthesizing a xeno-free and a feeder free self-renewal extracellular matrix for long-term maintenance of undifferentiated Human Embryonic Stem Cells (hESCs) and Human Pluripotent Stem Cells (hPSCs) comprises preparing a conditioned medium. The conditioned medium is prepared by adding a basal medium, a nutrition providing medium, a plurality of additives and a plurality of feeder cells together. The basal medium is neurobasal medium. The nutrition providing medium is Dulbecco's Modified Eagle Medium/F12 (DMEM/F12). The plurality of feeder cells are Human Dermal Fibroblasts (HDFs) which are inactivated by mitomycin C for 2 hours. The conditioned medium is incubated for a pre-determined amount of time at a pre-determined temperature. The pre-determined amount of time is 24 hours-72 hours. The pre-determined amount of temperature is 37° C. The plurality of additives includes L-glutamine (L-Gln), β-mercaptoethanol (β-ME), nonessential amino acids (NEAAs), and insulin-transferrin-selenite (ITS). The nonessential amino acids (NEAAs) are alanine, asparagines, aspartic acid, cysteine, cyctine, glutamine, glutathione, glycine, histidine, proline, serine, taurine and threonine. The extracellular matrix maintains an undifferentiated Human Embryonic Stem Cells (hESCs) and a Human Pluripotent Stem Cells (hPSCs) for a period of one year at room temperature. The extracellular matrix is in the form of a gel.

According to one embodiment herein, a method of synthesizing a culture plate provided with a coating of a xeno-free and a feeder free self-renewal extracellular matrix for long-term maintenance of undifferentiated Human Embryonic Stem Cells (hESCs) and Human Pluripotent Stem Cells (hPSCs) comprises coating a plate with a conditioned medium for a predetermined amount of time. The coating is simple coating. The coating is layer-by-layer coating. The predetermined amount of time is 5 min-15 min. The plates are coated with fractionated conditioned medium with an amount of >10 KD-30 KD.

According to another embodiment herein, a method of providing a culture plate with a simple coating of a xeno-free and a feeder free self-renewal extracellular matrix for long-term maintenance of undifferentiated Human Embryonic Stem Cells (hESCs) and Human Pluripotent Stem Cells (hPSCs) comprises adding the conditioned medium on a plate. The plate is swirled to evenly spread the conditioned medium. The plate is incubated at a predetermined temperature and for the predetermined amount of time. The pre-determined temperature is 37° C. The predetermined amount of time is 5 min-150 min. The plate is dried. The plate is dried at room temperature for 15-20 min. The simple coating comprises providing a single coating on the plate. The plates are coated with fractionated conditioned medium with 10 KD-30 KD

According to an embodiment herein, a method of providing a culture plate with a plurality of coatings of a xeno-free and a feeder free self-renewal extracellular matrix for long-term maintenance of undifferentiated Human Embryonic Stem Cells (hESCs) and Human Pluripotent Stem Cells (hPSCs) comprises adding the conditioned medium on a plate. The plate is swirled to evenly spread the conditioned medium. The plate is incubated at a predetermined temperature and for the predetermined amount of time. The pre-determined temperature is 37° C. The predetermined amount of time is 5 min and 150 min. The plate is dried. The plate is dried at room temperature for 15-20 min. The plate is recoated after drying for a plurality of times. The plurality of coatings is layer-by-layer coatings on the culture plate. The plates are coated with >10 KD and >30 KD fractionated conditioned medium.

According to one embodiment herein, the conditioned medium may be further added with a Rho-associated Coiled Kinase (ROCK) inhibitor Y-27632. The addition of ROCKi to ECM increases the plating and cloning efficiency.

The hPSCs are potentially promising for many therapeutic applications such as regenerative medicine and drug discovery. The hPSCs have the ability to continuously self-renew and differentiate into all specialized cell types. An important challenging component of the hPSC culture system is the ECM. The ECM is the extracellular matrix which is utilized to grow hPSCs. The ECM molecules are large, complex and often multimeric structures. The recombinant production of ECM is generally difficult or not cost effective.

A feeder free culture is a culture that does not contain any feeder cells. The feeder cells deliver an unknown collection of factors that allow hESCs to preserve their “stemness” and remain undifferentiated. But the presence of the feeder cells poses a real risk of transferring animal or human viruses to hESCs. The embodiments herein provide a feeder free ECM that does not provide any harm to the hESCs.

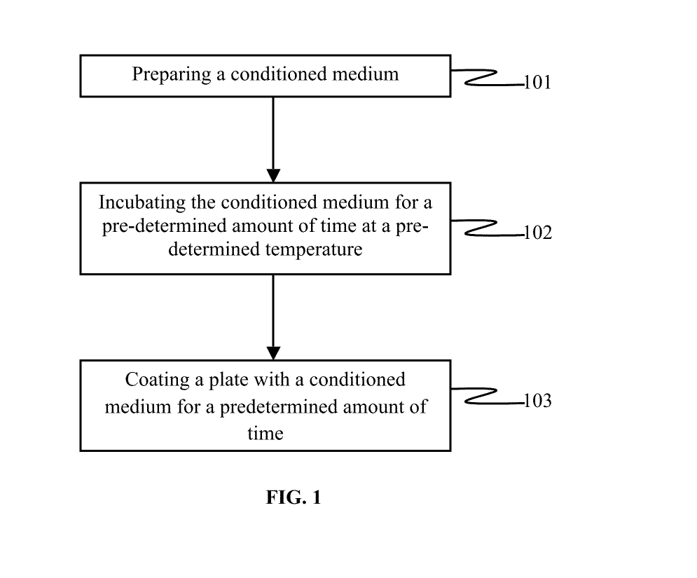

FIG. 1 shows a flow chart showing the various steps of a method of synthesizing a xeno-free and a feeder free self-renewal extracellular matrix for long-term maintenance of undifferentiated Human Embryonic Stem Cells (hESCs) and Human Pluripotent Stem Cells (hPSCs), according to an embodiment herein. With respect to FIG. 1, a conditioned medium is prepared (101). The conditioned medium is prepared by adding a basal medium, a nutrition providing medium, a plurality of additives and a plurality of feeder cells together. The basal medium is the neurobasal medium. The nutrition providing medium is Dulbecco's Modified Eagle Medium/F12 (DMEM/F12). The plurality of feeder cells are Human Dermal Fibroblasts (HDFs) inactivated by mitomycin C. The Human Dermal Fibroblasts (HDFs) are inactivated by mitomycin C for 2 hours. The conditioned medium is incubated for a pre-determined amount of time at a pre-determined temperature (102). The pre-determined amount of time is 24 hours and 72 hours. The pre-determined amount of temperature is 37° C. The plurality of additives includes L-glutamine (L-Gln), β-mercaptoethanol (β-ME), nonessential amino acids (NEAAs), and insulin-transferrin-selenite (ITS). The nonessential amino acids (NEAAs) are alanine, asparagines, aspartic acid, cysteine, cyctine, glutamine, glutathione, glycine, histidine, proline, serine, taurine and threonine. The extracellular matrix maintains the undifferentiated Human Embryonic Stem Cells (hESCs) and Human Pluripotent Stem Cells (hPSCs) for a period of one year at room temperature. The extracellular matrix is in the form of a gel. A plate is coated with the conditioned medium for a predetermined amount of time (103). The predetermined amount of time is 5 min and 15 min. The coating is simple coating. The coating is layer-by-layer coating.

According to one embedment herein, the simple coating comprises adding the conditioned medium on a plate and swirling the plate to evenly spread the conditioned medium. The plate is incubated at a predetermined temperature for the predetermined amount of time. The pre-determined temperature is 37° C. The predetermined amount of time is 5 min and 15 min. The plate is dried at room temperature for 15-20 min.

According to another embodiment herein, a layer-by-layer coating comprises adding the conditioned medium on a plate and swirling the plate to evenly spread the conditioned medium. The plate is incubated at a predetermined temperature for the predetermined amount of time. The pre-determined temperature is 37° C. The predetermined amount of time is 5 min and 15 min. The plate is dried at room temperature for 15-20 min. The plate is recoated after drying following the above steps for a plurality of times.

According to the embodiments herein, an extracellular matrix based on conditioned medium of human fibroblasts under serum-free and xeno-free culture conditions is provided. The extracellular matrix is developed in-house and is simple, robust, repeatable and cost effective. The embodiments herein provide a favorable surface environment for the Human pluripotent stem cells culture in the form of a conditioned medium. The embodiments herein also provide an optimized conditioned medium that develops undifferentiated cells which are similar to the conventional Extracellular Matrix or Matrigel (MG). The freeze/thaw protocol can be readily performed on this surface. The coated plates of the xeno-free substrate or ECM can be preserved for one year at room temperature while maintaining the capability for hPSC expansion. The ECM has the potential for large-scale expansion of hPSCs while providing an attractive cell culture platform for the production of cells for both research and future therapeutic applications.

### Experimental Details

### Materials and Methods

Culturing hESCs and hiPSCs:

The hESC lines i.e. Royan H5 and Royan H6 and hiPSC lines i.e. R1-hiPSC1 and R1-hiPSC4 were used in this study. Prior to this study the cell lines were passaged and maintained on Matrigel (MG) with 0.3 mg/ml concentration (Sigma-Aldrich, E1270) under feeder-free culture conditions in an hESC medium. The medium comprised DMEM/F12 medium i.e. Dulbecco's Modified Eagle Medium (Invitrogen, 21331-020) supplemented with 20% KockOut Serum Replacement (KOSR, Invitrogen, 10828-028) further with 2 Mm of L-glutamine (L-Gln, Invitrogen, 25030-024), 0.1 Mm of β-mercaptoethanol (β-ME, Sigma-Aldrich, M7522), 1% of nonessential amino acids (NEAAs, Invitrogen, 11140-035), 1% of penicillin and streptomycin (Invitrogen, 15070-063), 1% of insulin-transferrin-selenite (ITS, Invitrogen, 41400-045) and 100 ng/ml of basic fibroblast growth factor (bFGF, Royan Institute). The cells were grown in 5% Carbon dioxide (CO₂) at 95% humidity.

### Experimental Design

The parameters and experimental design for the development of an Extra Cellular Matrix (ECM) for the long-term culture of undifferentiated hESCs and hiPSCs are described in Table 1.

TABLE 1
Parameters and Experimental design for development of an ECM
“RoGel”

| S. No. | Parameter | Variable |
|---|---|---|
| 1. | Conditioned Medium (CM) | Neurobasal medium |
| | | DMEM/F12 medium |
| 2. | Cell density of mitomycin C | 5000 cell/cm² (Low Density) |
| | inactivated Human Dermal | 50000 cell/cm² (High Density) |
| | Fibroblasts (HDF) to prepare | |
| | CM | |
| 3. | Time of conditioning of CM | 24 h |
| | | 72 h |
| 4. | Time of coating | 5 min |
| | | 150 min |
| 5. | Method of coating | Simple |
| | | Layer by layer |
| 6. | ROCK inhibitor (Y-27632) | Presence in coating |
| | | Absence in coating |
| 7. | Dilution rate of CM | Pure CM |
| | | Half diluted CM |

The Matrigel (MG) was used as a positive control in all experiments. In the first phase, with respect to table 1, the Royan H5 cell line was cultured in CM coated plates (six-well, TPP, 92006). The CM was prepared by either 24 h or 72 h incubation of neurobasal medium (Invitrogen, 21103049) supplemented with L-Gln, β-ME, NEAAs and ITS on human dermal fibroblast (HDFs) feeder layers. The HDFs were inactivated by mitomycin C for 2 h. The mitomycin C was used with a concentration of 10 μg/ml (Sigma-Aldrich, M0503). The feeder cells were cultured at high densities and at low densities in 25 cm² T flasks (TPP, 90025) in 4 ml of conditioned medium (160 μl/cm²). The high density comprised of 50000 cells/cm² and the low density comprised of 5000 cells/cm². The plates were exposed to CM for either 5 min or 150 min while coating. These conditions were compared using a plating efficiency (PE) on the seventh day after passage (number of colonies/number of seeded clusters×100).

After the selection of a comparable group with MG in the first phase, Royan H5 cells were cultured in pure or 50% diluted CM in the presence or absence of ROCK inhibitor, 1 μM of Y-27632 dihydrochloride monohydrate (Sigma-Aldrich, Y0503). It is recently found that the plating efficiency increased from 1.5 to 2 fold in the groups that contained a ROCK inhibitor in MG compared to those that contained ROCK inhibitor in a culture medium only, which was also reported by Watanabe et al. It was observed in the present experiment that the similar plating efficiencies were observed using a lower concentration of Y-27632 i.e. 800 Nm=EC50 when compared to a higher concentration of Y-27632 i.e. 10 Mm. The plates were then coated using a simple or layer-by-layer coating method in the second phase.

To coat each well of a six-well plate, 1 ml CM was added and swirled to spread the CM across the surface. The plates were then incubated at 37° C. After 150 min, the extra CM was removed and allowed to dry at room temperature for about 15-20 min. This type of procedure was called “simple coating”. To have layer-by-layer coating, the plates were recoated again using the same procedure after the first coating was allowed to dry. It was noted that before cell seeding the plates were rinsed by Dulbecc's phosphate-buffered saline that contained Ca²⁺ and Mg²⁺ ions (DPBS, Invitrogen, 14040-117).

Grading Scale of hESCs and hiPSCs:

To determine the impact of culture conditions on the undifferentiated growth of cells the quality of the morphologies of the grown colonies was evaluated after seven days of passaging. The colonies were assessed using an inverted microscope set at 10× magnification (Olympus, CKX41, Tokyo, Japan). The quality of the colonies was scored as previously determined.

Briefly, the colony quality was graded as “A” i.e. excellent when the colonies had even morphology and well-defined edges. The cells in grade “A” colonies were dense and individual cells could not be easily distinguished. The grade “A” colonies were thick, multilayered, homogenous and exhibited 0%-30% differentiation. The differentiated cells migrated and passed from the periphery of the colonies. The second category was “B” i.e. good where 30%-50% of the peripheral area was differentiated. The third category was “C” i.e. fair in which the colonies exhibited more than 50%-80% of differentiation and the forth category was “D” i.e. poor in which the colonies differentiated more than 80%-100% of differentiation and exhibited well-differentiated morphologies with inhomogeneous levels.

Passaging of Human Pluripotent Stem Cells (hPSCs):

For passaging, the cells were washed once with DPBS that contained Ca²⁺ and Mg²⁺ ions and then incubated with DMEM/F12 that consisted of 1:1 collagenase IV with 1 mg/ml concentration (Invitrogen, 17104-019) and dispase with 2 mg/ml concentration (Invitrogen, 17105-041) at 37° C. for 3-5 min. When the colonies at the edge began to dissociate from the bottom, the enzyme was removed and plates were twice washed with DPBS that contained Ca²⁺ and Mg²⁺ ions. The cells were collected by gentle pipeting and then re-plated on MG (Sigma-Aldrich, E1270) or CM-coated vessels. The medium was changed every other day.

Immuno Fluorescence and Alkaline Phosphatase (ALK) Staining:

The cells were fixed in 4% paraformaldehyde for 20 min. The cells were then permeabilized with 0.2% triton X-100 for 30 min and blocked in 10% goat serum in PBS for 1 h. The cells were incubated with the appropriate primary antibody for 1 h at 37° C. and washed with PBS and incubated with FITC-conjugated secondary antibodies, anti-mouse IgM (1:100, Sigma-Aldrich, F9259), anti-rat IgM (1:200, eBioscience, 11-0990) and anti-mouse IgG (1:200, Sigma-Aldrich, F9006) as appropriate for 30 min at 37° C. The primary antibodies were anti TRA-1-81 (1:100, Chemicon, MAB4381), Oct4 (1:100, Santa Cruz Biotechnology, SC-5279) and SSEA-3 (1:50, Chemicon, MAB4303) for undifferentiated hESC and hiPSC determination. The nuclei were counterstained with DAPI with 0.1 μg/ml concentration (Sigma-Aldrich, D8417). The cells were analyzed with a fluorescent microscope (Olympus, Japan).

The Alkaline phosphatase staining was performed based on the manufacturer's recommendations (Sigma-Aldrich, 86R). The stained colonies on MG and CM coated vessels were captured using a digital camera (Canon, IXUS 950 IS) and photos were analyzed by ImageJ software version 1.4.

Flow Cytometric Analysis:

The Ki-67 antigen which is an excellent marker to exhibit cell proliferation of hPSCs cultured on different substrates was analyzed by flow cytometry using the monoclonal mouse anti-human Ki-67 antigen, clone Ki-67 (F0788, Dako Cytomation). For the purpose of quantization of the pluripotent status of hESCs after culture in various conditions i.e. two different substrates combined with two different media, the cells were analyzed by flow cytometry for NANOG (1:500, Sigma-Aldrich, N3038). The matched secondary antibody was Alexa Fluor® 488 goat anti-mouse IgG (1:500, Invitrogen, A-11001).

Briefly, hPSCs cultured on 6 cm dishes (BD Falcon™ 60 mm Easy-Grip™ Cell Culture Dish, BD, 353004) were coated with CM or MG (for Ki-67) in hESC or mTeR1 media (for NANOG). The cells were harvested in single cell format by treatment with trypsin/EDTA and then fixed with ice cold methanol, blocked by 2% normal goat serum for 60 min at room temperature, washed, incubated with primary antibody for 1 h at 37° C. and washed again. The cells were then treated with secondary antibody for 30 min at 37° C. (Ki-67 is directly conjugate with FITC), washed and finally analyzed by flow cytometry. As a negative control, the cells were stained with the appropriate isotype-matched control. The Flow cytometric analysis was performed using a FACSCalibur Flow Cytometer (BD Biosciences) and the acquired data was analyzed using BD CellQuest™ Pro software (Version 0.3.cfab).

Karyotype Analysis:

For karyotype analysis, the cells were prepared as described by Mollamohammadi et al.

Analysis of Gene Expression:

Total RNA was isolated using the Nucleospin Kit (MN) and treated with a Dnasel Rnase Free Kit (Fermentas) in order to remove genomic DNA contamination. 2 μg of total RNA for a reverse transcription reaction with the RevertAid First Strand Cdna Synthesis Kit (Fermentas) and a random hexamer primer was used according to the manufacturer's instructions. The sequences of primers are presented in Table 2 and Totonchi et al.

TABLE 2
Primers and reaction conditions used in RT-PCR

| | Primer sequences | Length | Annealing | Accession |
|---|---|---|---|---|
| Genes | (5′-3′) | (bp) | temp.(° C.) | number |
| GAPDH | CTCATTTCCTGGTATGACAACGA | 121 | 60 | NM_0020 |
| | CTTCCTCTTGTGCTCTTGCT | | | 46.3 |
| OCT 4 | CTGGGTTGATCCTCGGACCT | 128 | 60 | NM_0027 |
| | CACAGAACTCATACGGCGGG | | | 01.4 |
| NANOG | AAAGAATCTTCACCTATGCC | 110 | 60 | NM_0248 |
| | GAAGGAAGAGGAGAGACAGT | | | 65.2 |
| KLF4 | ATTACCAAGAGCTCATGCCA | 152 | 61 | NM_0042 |
| | CCTTGAGATGGGAACTCTTTG | | | 35.3 |
| SOX2 | GGGAAATGGGAGGGGTGCA | 151 | 60 | NM_0031 |
| | AAAGAGG | | | 06.2 |
| | TTGCGTGAGTGTGGATGGG | | | |
| | ATTGGTG | | | |
| PAX6 | GTCCATCTTTGCTTGGGAAA | 110 | 60 | NM_0011 |
| | TAGCCAGGTTGCGAAGAACT | | | 27612.1 |
| Nestin | TCCAGGAACGGAAAATCAAG | 120 | 60 | NM_0066 |
| | GCCTCCTCATCCCCTACTTC | | | 17.1 |
| Brachyury | AATTGGTCCAGCCTTGGAAT | 112 | 60 | NM_0031 |
| | CGTTGCTCACAGACCACA | | | 81.2 |
| GATA4 | CCTGTCATCTCACTACGG | 180 | 60 | NM_0020 |
| | GCTGTTCCAAGAGTCCTG | | | 52.3< |
| ALB | CTTCCTGGGCATGTTTTTGT | 140 | 60 | NM_0004 |
| | TGGCATAGCATTCATGAGGA | | | 77.5 |
| FOXA2 | ATGCACTCGGCTTCCAGTAT | 120 | 60 | NM_0104 |
| | TGTTGCTCACGGAGGAGTAG | | | 46 |

In-Vitro Differentiation:

To demonstrate whether the cultured Hesc and hiPSC colonies on CM-coated dishes were pluripotent, the colonies were assayed for their ability to differentiate into lineages representative of the three embryonic germ layers by embryoid body (EB) formation and spontaneous differentiation as described in Mollamohammadi et al.

Teratoma Formation Assay:

About 2-3×10⁶ hPSCs with undifferentiated morphology were collected by treatment with trypsin/EDTA (1×, Invitrogen, 25300-054). The treated hPSCs were mixed in 40-60 μL MG and injected into the testes of 5-8 week-old nude mice. Teratomas formed approximately 12 weeks after injection and were surgically removed and fixed with Bouin's fixative for five days at room temperature, then embedded in paraffin. Sections were cut at a thickness of 6 μM, processed with hematoxylin and eosin staining and observed under a brightfield microscope.

Colony Formation of Single Dissociated hESCs and hiPSCs:

To evaluate the effect of CM-coated dishes versus MG-coated dishes on the cloning efficiency of single dissociated hESCs and hiPSCs [(number of ALP⁺ colonies/number of single dissociated seeded cells)×100], the number of feeder-independent colonies of dissociated single hPSCs were analyzed.

For this purpose, hESCs and hiPSCs were dissociated as single cells by 0.05% trypsin/EDTA for 5 min at 37° C. and then collected by gentle pipeting. The cells were cultured on CM-coated dishes and MG-coated dishes. After seven days colonies were stained using an ALP kit, captured and photos were analyzed with ImageJ.

Cryopreservation of hESCs and hiPSCs:

The hESCs and hiPSCs were frozen as previously described by Baharvand et al., with some modifications. Briefly, the hESCs and hiPSCs were trypsinized as single cells and subsequently collected by gentle pipeting. The single dissociated cells were frozen in 10% dimethyl sulfoxide (DMSO, Sigma-Aldrich, D2650) plus 90% KnockOut Serum Replacement (KOSR), at aliquots of 1×10⁶ cells per 250 μl ice-cooled freezing medium. The cryovials were transferred to a −80° C. freezer overnight and then into a liquid nitrogen tank the following day for long-term storage.

Fractionation of CM:

To determine the effective fraction of CM in undifferentiated growths of hESCs and hiPSCs, the Millipore's Amicon Ultra-15 centrifugal filter devices was used (10K, UFC9 010 08 and 30K, UFC9 030 08, Millipore) based on the molecular weights of the CM proteins (MWCO). Purification was performed based on the manufacturer's recommendations. Briefly, 12 Ml of CM was added to the Amicon Ultra 10K device and centrifuged at 5,000×g (maximum) for approximately 15-60 min. Next >10 KD and <10 KD fractionated CM were separately coated on culture dishes. The same process was performed by an Amicon Ultra 30K device for the production of >30 KD and <30 KD fractionated CM-coated culture dishes. The hESCs i.e. Royan H6 at passage 12 were cultured on these four fractions of CM for additional analysis.

Statistical Analysis:

The plating efficiency (PE) and grading morphology were conducted in nine replications. About 20 clamps were used per replicate in each group (20 clamps/well of a six-well plate). Results were expressed as mean±standard deviation (SD). The lines were characterized after 11-16 passages in different conditions. Plating, cloning efficiency and morphology grading were compared using Kruskal Wallis-Mann Whitney or one-way ANOVA followed by Tukey's post hoc test. The PE (based on the ratio of undifferentiated ALK-positive colonies formed per initially seeded hESCs and hiPSCs clumps) was calculated under phase contrast inverted microscope (CKX 41, Olympus). The cloning efficiency (based on the ratio of ALP-positive colonies formed per initially seeded hESCs and hiPSCs single cells) was calculated by ImageJ software version 1.4. Cloning efficiency assay was replicated three times. The mean difference was significant at p<0.05.

### Results and Discussion

Determining the Type of Diluting Medium Used for Matrigel, Feeder Cell Type and their Densities:

Preparation of an “optimized Conditioned Medium (CM)”: Initially, it was examined that whether the DMEM/F12 or the neurobasal medium could support the attachment of more hPSCs in the course of Conditioned Medium (CM) preparation as a fibroblast culture medium. The efficient impact of neurobasal medium on the maintenance of hPSCs on a suspension culture has been previously described by Steiner et al. Therefore it was decided to prepare CM with a DMEM/F12 or neurobasal medium on low density HDF for a 24 h culture. The Matrigel (MG) was diluted in DMEM/F12 as a conventional medium for hESC culture or neurobasal medium. The plates were then coated for 150 min. Cell attachment was assessed after seven days as Plating Efficiency (PE) using Royan H5 hESCs lines.

$$PE = \frac{No . ofALPpositivecolonies}{No . ofseededexplants} \times 100$$

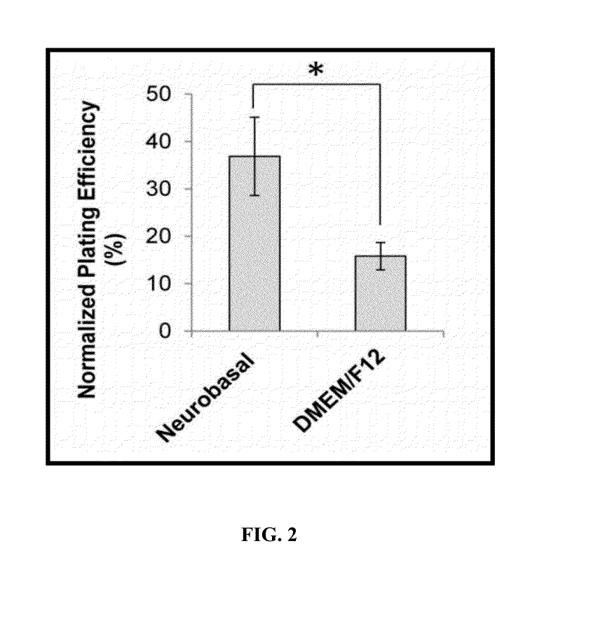

FIG. 2 shows a histogram showing the Normalized plating efficiency (PE) of the neurobasal medium and DMEM/F12 medium on the attachment of hPSCs, according to the embodiments herein. With respect to FIG. 2, it was found that the neurobasal medium was more effective for the attachment of hPSCs with p<0.001. The plating efficiency (PE) was significantly higher on MG diluted with neurobasal medium as compared to DMEM/F12.

Afterwards, the six-well plates were coated for either 5 min or 150 min. The serum free-neurobasal CM at this step was derived from high density and low density Human Dermal Fibroblasts (HDFs) for 24 h and 72 h in a neurobasal medium supplemented with L-glutamine (L-Gln), β-mercaptoethanol (β-ME), nonessential amino acids (NEAAs) and insulin-transferrin-selenite (ITS) without addition of serum or kockout serum replacement (KOSR).

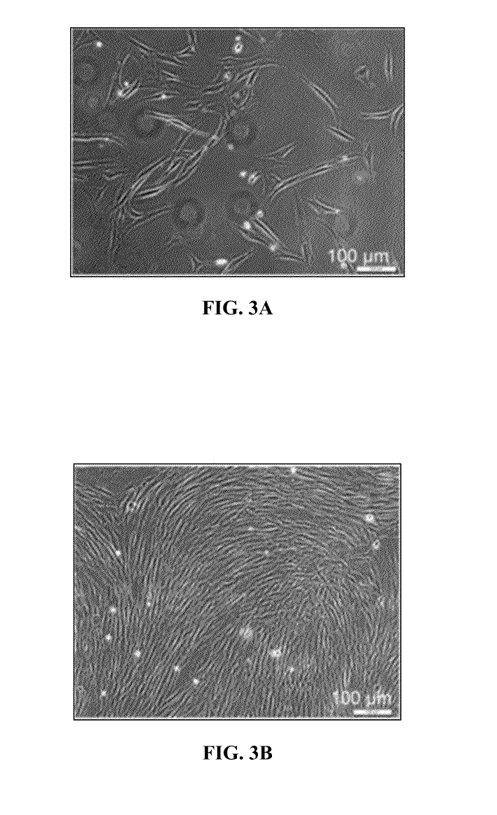

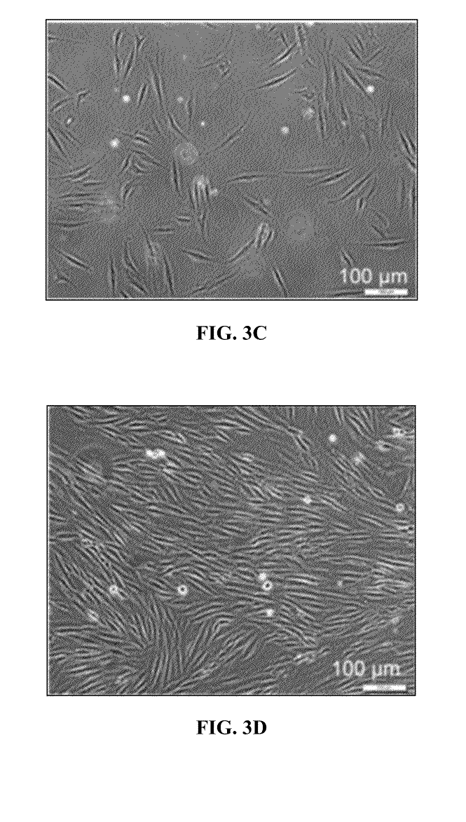

FIG. 3A shows an image showing the feeder cells derived from Human Dermal Fibroblasts (HDFs) at a low density, according to the embodiments herein. With respect to FIG. 3A, growth of HDFs can be seen. The scale is 100 μm. The cell density is 5×10³ cell/cm².

FIG. 3B shows an image showing the feeder cells derived from Human Dermal Fibroblasts (HDFs) at a high density, according to the embodiments herein. With respect to FIG. 3B, growth of HDFs can be seen. The scale is 100 μm. The cell density is 50×10³ cell/cm².

FIG. 3C shows an image showing the feeder cells derived from Human Foreskin Fibroblasts (HFFs) at a low density, according to the embodiments herein. With respect to FIG. 3C, growth of HFFs can be seen. The scale is 100 μm. The cell density is 5×10³ cell/cm².

FIG. 3D shows an image showing the feeder cells derived from Human Foreskin Fibroblasts (HFFs) at a high density, according to the embodiments herein. With respect to FIG. 3D, growth of HFFs can be seen. The scale is 100 μm. The cell density is 50×10³ cell/cm².

Determination of Plating Efficiency of hPSCs Plated on Conditioned Medium Under Different Conditions to Develop an “RoGel”:

The plating efficiency (PE) is calculated as:

$$PE = \frac{No . ofALPpositivecolonies}{No . ofseededexplants} \times 100$$

The Conditioned medium (CM) was prepared from neurobasal medium. Royan H5 hESCs were cultured for seven days in these conditions. The Royan H5 hESCs were seeded into six-well coated plates and cultured for seven days in a hESC medium. MG-coated six-well plates were used as positive controls.

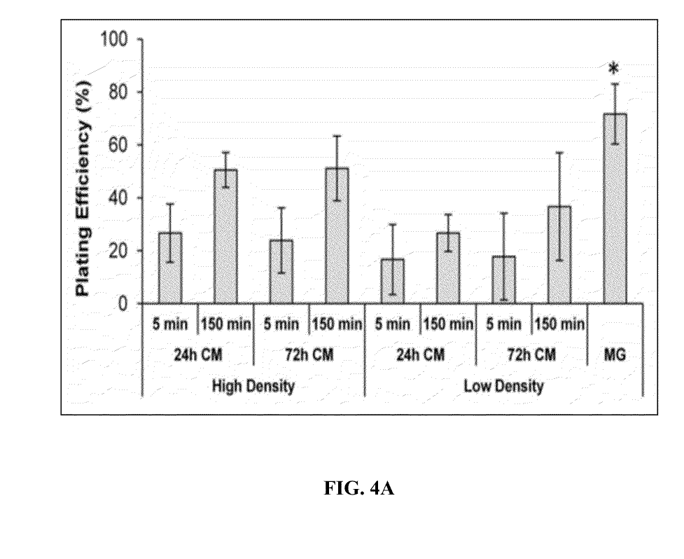

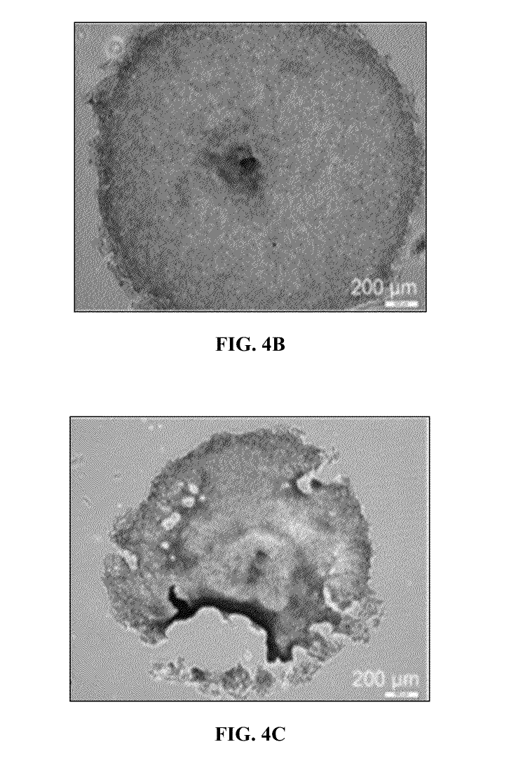

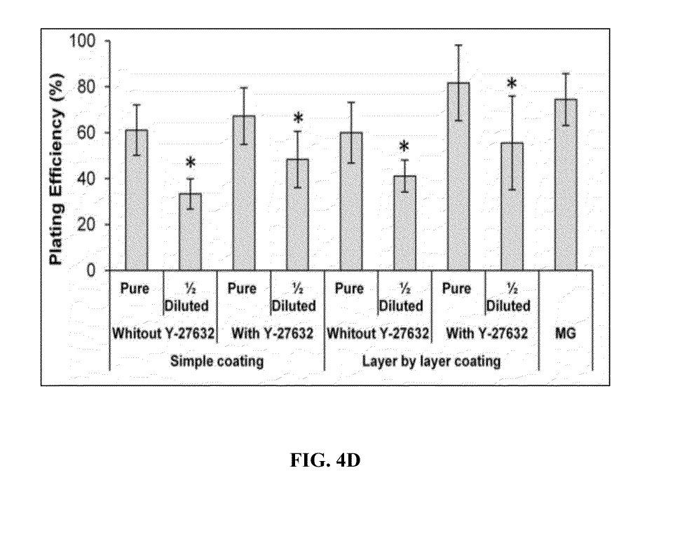

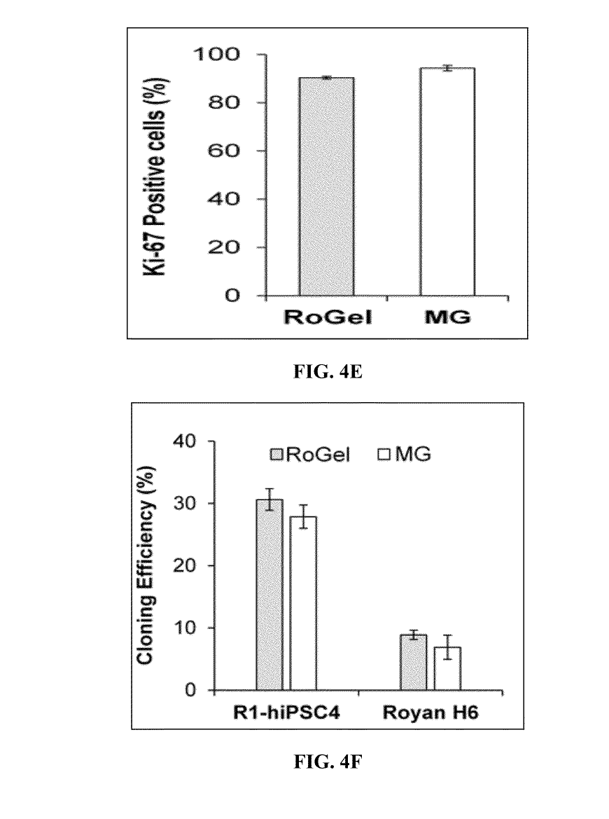

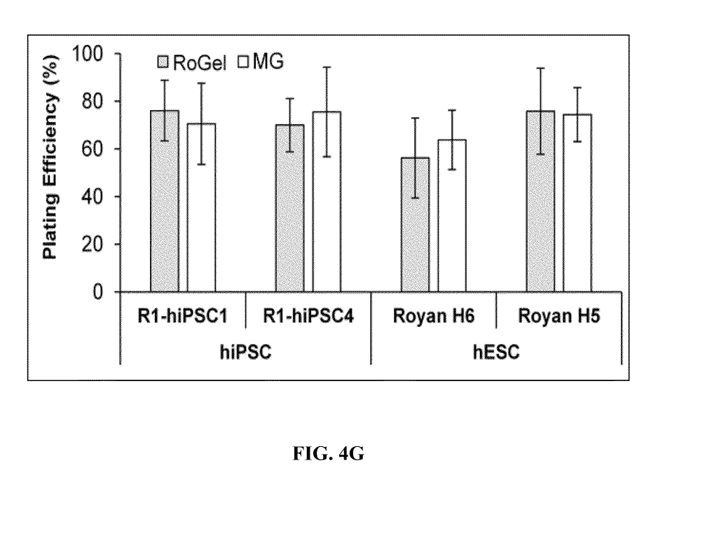

FIG. 4A shows a histogram showing the plating efficiency of hPSCs plated on conditioned medium under different conditions such as cell density, time of conditioning and coating time, according to the embodiments herein. The cell density was high and low. The high cell density was 50000 cells/cm² and the low density was 5000 cells/cm². The time of conditioning was 24 h and 72 h. The time of coating was 5 min and 15 min. With respect to FIG. 4A, the high density, 24 h of CM or 72 h CM conditioning and 150 min of coating conditions resulted in insignificant PE compared to Matrigel (MG) i.e. at least p<0.05 MG vs other groups. The high density HDF derived after 72 h CM conditioning that were coated for 150 min supported the adhesion and morphology of hESCs to a similar extent as MG as shown in FIG. 2. The adhesion and the morphology are evaluated by the ratio of the tightly packed colonies with a high cell nucleus: cytoplasm.

FIG. 4B shows an image of a dish showing a growth of hPSCs stained with Alkaline phosphatase stain in the conditioned medium (CM) with high cell density and conditioned for 72 h, according to the embodiments herein. With respect to FIG. 4B, the cells in 72 h CM adhered more tightly to the dish. The scale of the image is 200 μm.

FIG. 4C shows an image of a dish showing a growth of hPSCs stained with Alkaline phosphatase stain in the conditioned medium with high cell density conditioned for 24 h, according to the embodiments herein. With respect to FIG. 4C, it can be seen that the cell adhesion is less. The colony roll back and edge detachments were seen but the hESC colonies were more tightly attached. The scale of the image is 200 μm. Since, the cells in 72 h CM adhered more tightly to the dish therefore these conditions were selected for the next experiment.

For further optimization of CM, the hESCs were cultured in six-well plates coated with pure or half-diluted CM with fresh medium in the presence or absence of ROCKi. The ROCKi was added in the CM because it was discovered earlier that the addition of ROCKi to ECM here MG increased plating and cloning efficiency (number of colonies per seeded single hPSCs) more than when it was added to medium. The wells were coated by both adding CM and swirling the plates to spread the CM solution across the surface or by using layer-by-layer repetition of the previous coating after allowing the first coat to dry at room temperature.

FIG. 4D shows a histogram showing the effect of dilution of the CM in presence and absence of ROCKi with a presence and absence of Y-276320 in coating the CM and the effect of different coating method, according to the embodiments herein. The different coating methods are simple coating and layer-by-layer coating. With respect to FIG. 4D, the culture of cell clumps in pure CM in presence of ROCKi in the coating CM and made by a layer-by-layer method of coating resulted in a plating efficiency similar to the plating efficiency of MatriGel alone with atleast p<0.05 vs MG. Although some hESC colonies were found attached to the diluted CM in the absence of ROCKi but based on the quality of colonies and the attachment present in the pure CM and ROCKi in both the simple coating and layer-by-layer methods for CM coating enabled better hESC PE.

The flow cytometry test for Ki67 revealed that the proliferation of hESC was similar to hESCs that were cultured on MG. Ki67 acts as a marker of proliferating cells. FIG. 4E shows a histogram showing the percentage of Ki-67 positive cells for the Rogel and the matrigel, according to the embodiments herein. The Rogel is the conditioned medium prepared according to the embodiments herein. With respect to FIG. 4E, the cellular proliferation on CM and MG indicated similar Ki67-positive cells.

Given the role of ECM in cloning efficiency, the number of colonies of dissociated single Royan H6 hESCs and R1-hiPSC4 were analyzed. For this purpose, the hPSCs were seeded at a density of 5×10⁴/well in six-well plates and allowed to grow for seven days and compared. FIG. 4F shows a histogram showing the cloning efficiency of the R1-hiPSC4 and Royan H6 grown in the CM and MG, according to the embodiments herein. The cloning efficiency is calculated as:

$$CloningEfficiency = \frac{No . ofALP + colonies}{No . ofsingleseededdissociatedcells} \times 100$$

With respect to FIG. 4F, the number of colonies formed after seeding in CM was similar when compared to MG coated plates that contained ROCKi.

Thus according to the embodiments herein, the conditioned medium supplemented with L-Gln, β-ME, NEAAs and ITS on high density human fibroblast feeder, conditioned for 72 h was used as a ECM for hPSCs. According to another embodiment herein, the culture dishes for culturing hPSCs comprises atleast one coating of the pure conditioned medium added with ROCKi. The coating is done for atleast 150 min. The method of coating is layer-by-layer method. The ROCKi was added with a concentration of 800 nM. The in-house prepared ECM was named as “RoGel”.

The universality of “RoGel” according to the embodiments herein was analyzed for different cell lines. The Rogel comprising a pure CM having neurobasal medium on high density feeder, conditioned for 72 h and coated for 150 min by layer-by-layer method in the present of ROCKi was tested for two hESC and two hiPSC lines. FIG. 4G shows a histogram showing the plating efficiency of the grown hESC and hiPSC cell lines on the CM and the MG, according to the embodiments herein. The hESC cell lines are Royan H6 and Royan H5 while the hiPSC cell lines are R1-hiPSC1 and R1-hiPSC4. With respect to FIG. 4G, the similar results were obtained for Royan H6 hESCs, R1-hiPSC1 and R1-hiPSC4 on RoGel. The experiments were conducted with either Royan H5 or Royan H6 hESCs. The results obtained were similar to that of the MG. Thus the conditioned medium synthesized according to the embodiments herein is suitable for a long term growth of the Human Fibroblast Stem Cells.

Effects of Source, Passage and Age of Feeder Cells on Quality of “RoGel” or the Conditioned Medium According to the Embodiments Herein:

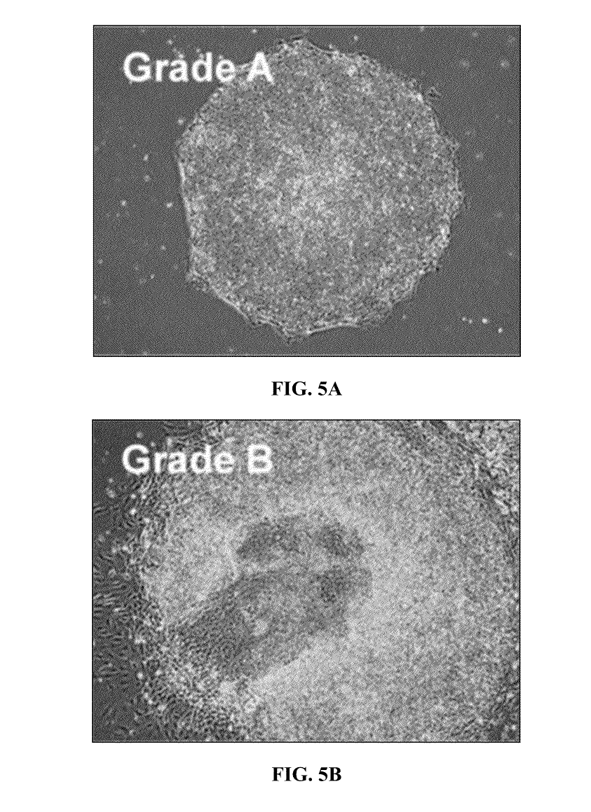

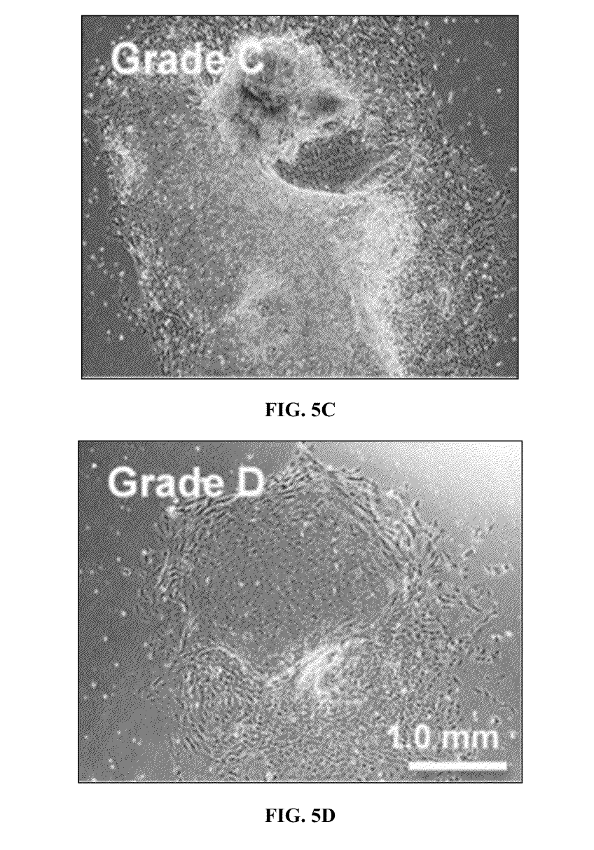

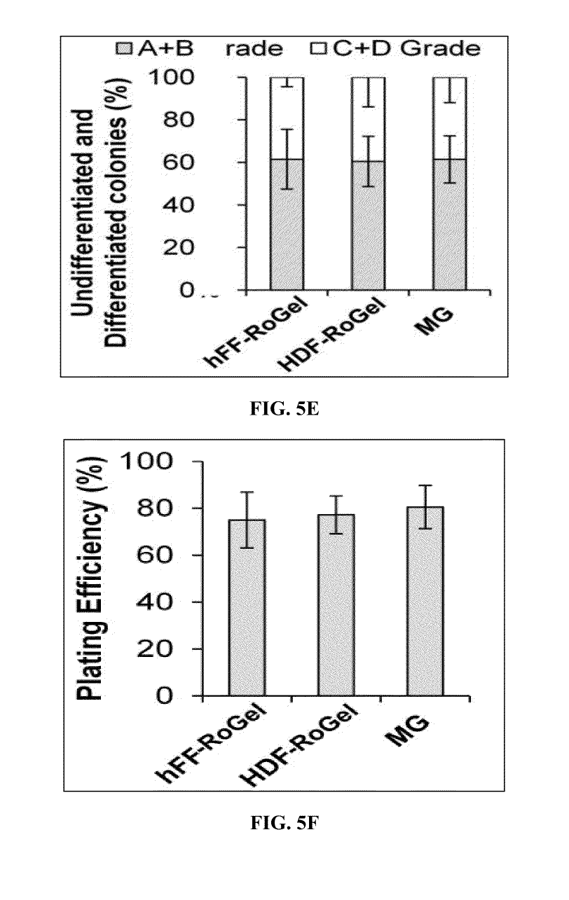

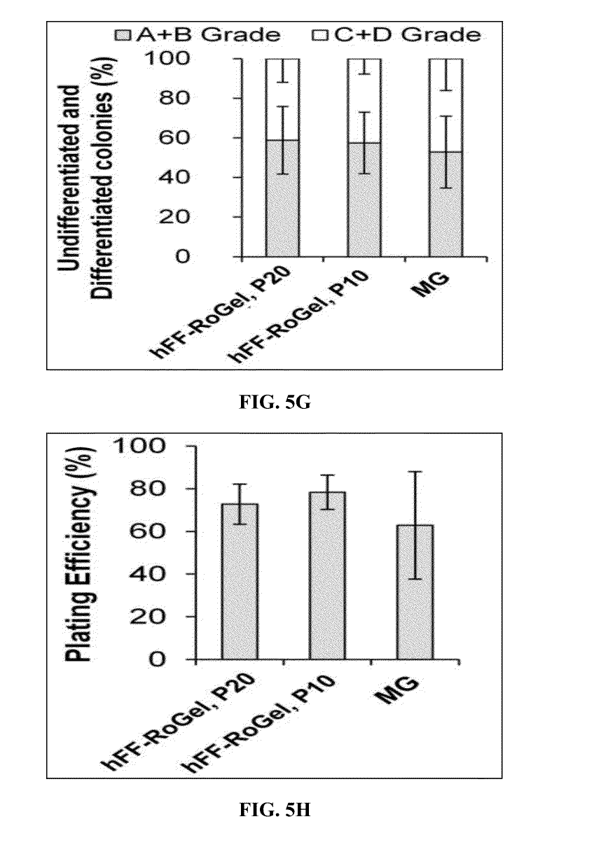

To evaluate the impact of feeder origin on the PE of the “RoGel” or the conditioned medium according to the embodiments herein, the ability of CM derived from HDF and HFF to support the self-renewal of Royan H5 hESCs was tested. The Royan H5 hESCs were cultured for seven days in these conditions. FIG. 5A-FIG. 5D are the images showing the growth morphology of the hPSC colonies post-subculturing, where FIG. 5A denotes grade-A, FIG. 5B denotes grade-B, FIG. 5C denotes grade-C and FIG. 5D denotes grade-D, according to the embodiments herein. With respect to FIG. 5A-FIG. 5D, the different growths of hPSC colonies were evaluated and denoted as grade A, B, C and D. With respect to FIG. 5D, the scale was kept 1.0 mm.

FIG. 5E shows a histogram showing the percentages of the undifferentiated and differentiated colonies in presence of HFF Rogel or conditioned medium derived from HFF as feeder cells and HDF Rogel or the conditioned medium derived from HDF as feeder cells and MG, according to the embodiments herein. With respect to FIG. 5E, the morphology qualities of the cultured colonies of hPSC lines were similar to MG.

FIG. 5F shows a histogram showing the Plating Efficiency of the cultured medium grown in presence of HFF Rogel or conditioned medium derived from HFF as feeder cells and HDF Rogel or the conditioned medium derived from HDF as feeder cells and MG, according to the embodiments herein. With respect to FIG. 5F, it was found that the plating efficiency in HFF-Rogel and HDF Rogel was similar to MG.

FIG. 5G shows a histogram showing the percentages of the undifferentiated and differentiated colonies in presence of HFF Rogel or conditioned medium derived from HFF as feeder cells and HDF Rogel or the conditioned medium derived from HDF as feeder cells at passage number 10 and passage number 20 and MG, according to the embodiments herein. The Passage number refers to the number of times the cells have been subcultured. With respect to FIG. 5G, the morphology qualities of the cultured colonies of hPSC lines were similar to MG.

FIG. 5H shows a histogram showing the Plating Efficiency of the cultured medium grown in presence of HFF Rogel or conditioned medium derived from HFF as feeder cells and HDF Rogel or the conditioned medium derived from HDF as feeder cells at passage number 10 and passage number 20 and MG, according to the embodiments herein. With respect to FIG. 5H, it was found that the plating efficiency in HFF-Rogel and HDF Rogel was similar to MG.

With respect to FIG. 5E, FIG. 5F, FIG. 5G and FIG. 5H, the percentage of different qualities by morphology of the cultured hPSC lines with CM derived from HDF or HFF or MG at their different passages of 10 or 20, it was found that Rogel derived from both types of feeder cells effectively supported self-renewal of hESCs. The quality of the colonies was similar in the “RoGel” of both feeders. The CM that was derived from different passages i.e. 10 or 20 of feeders supported similar hESC Plating Efficiency (PE) and quality when compared with MG.

To test whether the CM derived from cultured feeder cells at different times could influence the plating efficiency (PE) or not. For this purpose, the HDF feeder cells were cultured and CM was prepared after 1, 7, 14 and 28 weeks following the preparation of inactivated HDF by mitomycin C. FIG. 5I shows a histogram showing the effect of age of feeder cells on CM after the preparation of inactivated HDF by mitomycin C, according to the embodiments herein. With respect to FIG. 5I, similar plating efficiency was observed until about 14 weeks culture of inactivated HDF (p<0.005)(P<0.001, 1W vs other group).

Effect of Temperature and Time of CM Storage on Quality of “RoGel”:

To investigate the effect of temperature maintenance on the activity of the conditioned medium according to the embodiments herein, the CM was preserved at 4° C. or −20° C. for one week. The plates were then coated. The hESCs were seeded on the “RoGel”. The Royan H5 hESCs were cultured for seven days. To assess the expiration date of “RoGel” or the CM coated plates, the plates were maintained for 2, 4, 25, 32, 42 and 52 weeks at room temperature in a dark and dry place.

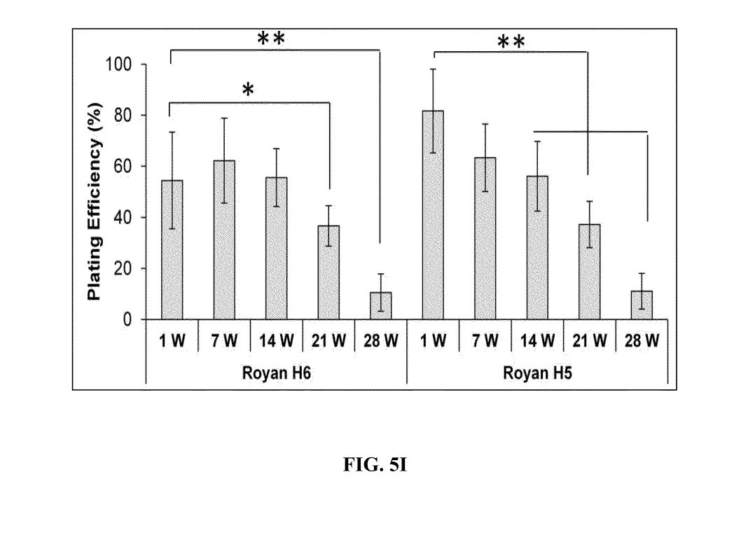

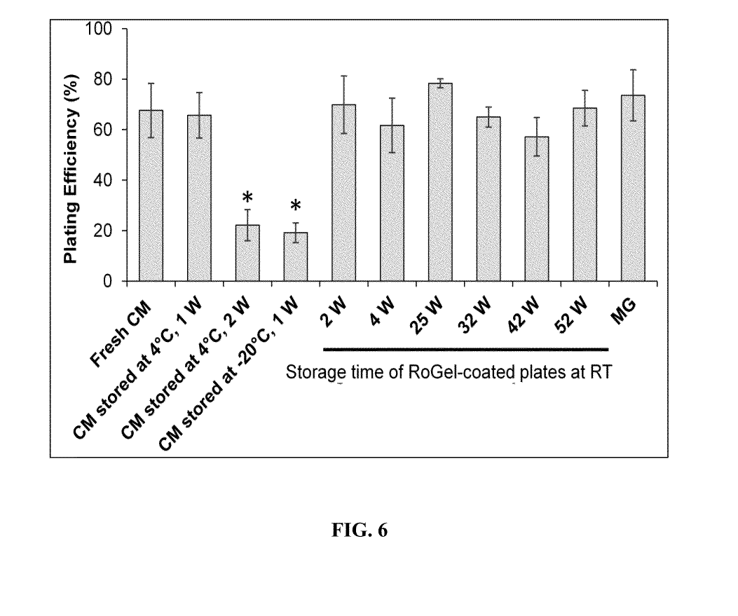

FIG. 6 shows a histogram showing the influence of temperature and time of the storage on the quality of CM, according to the embodiments herein. With respect to FIG. 6, it was observed that the Plating Efficiency of Royan H5 hESCs reduced significantly after freezing (p<0.001). Additionally, the Plating Efficiency of Royan H5 hESCs decreased significantly when the plates were coated by CM stored at 4° C. for two weeks (p<0.001). It was also found that the plates coated with “RoGel” were sufficient enough to yield Plating Efficiency after a year. The plates were still similar to MG control cultures and fresh “RoGel”.

Passaging and Freeze/Thaw of hPSCs on “RoGel”:

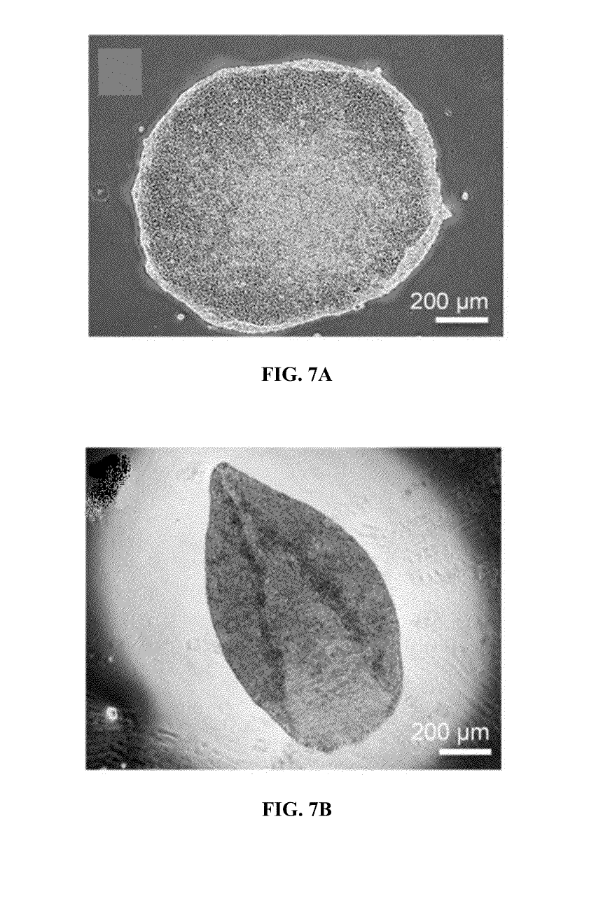

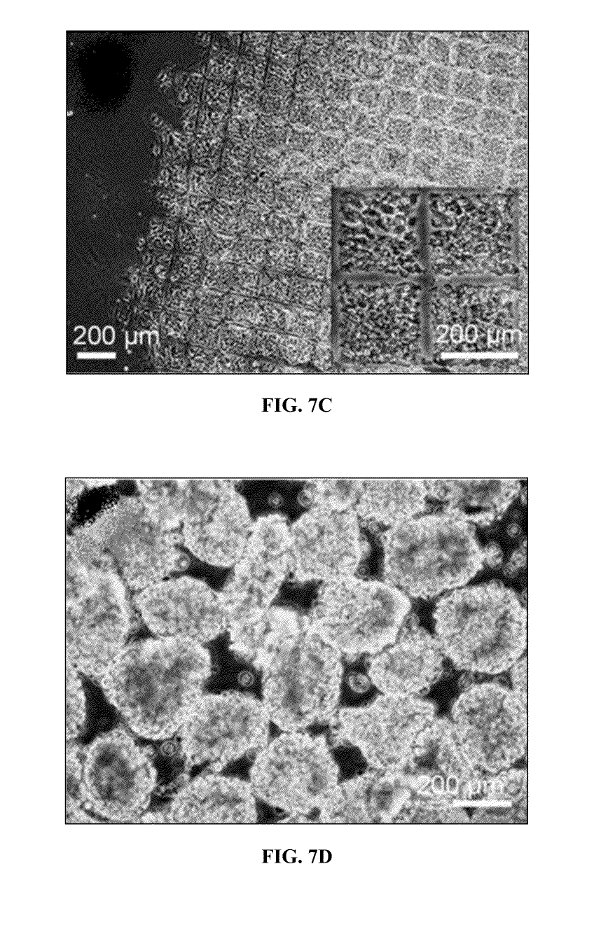

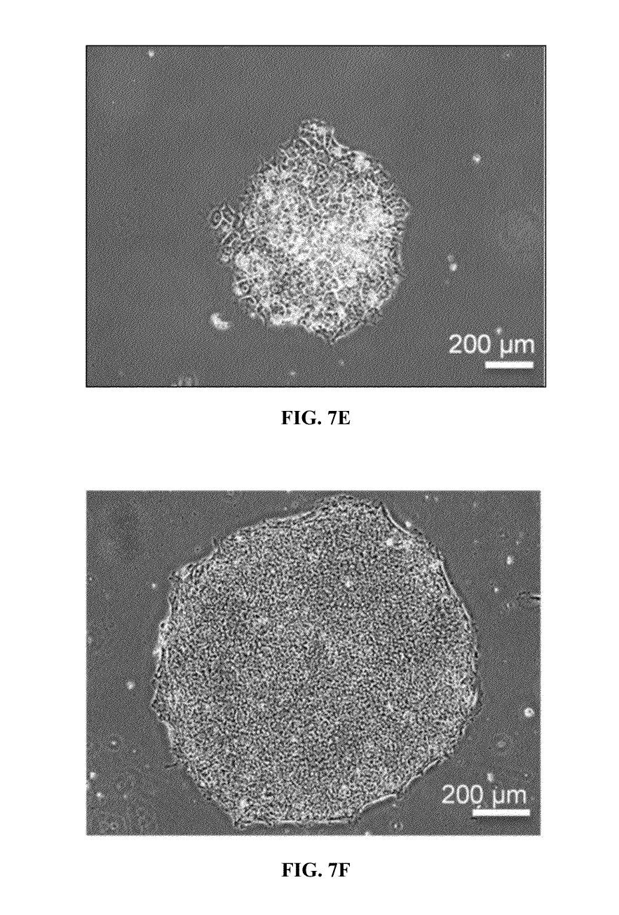

The cultured colonies on CM coated dishes were treated with enzymes. The cultures were treated with enzymes comprising trypsin/EDTA, dispase with 2 mg/ml concentration (Invitrogen, 17105-041) and StemPro® Accutase® Cell Dissociation Reagent (Invitrogen, A1110-501). FIG. 7A and FIG. 7B shows images of the colonies on CM coated dishes showing the effect of the enzymes treatment on the colonies where FIG. 7A shows the image of colony before the enzymatic treatment and FIG. 7B shows an image of the colony after the enzymatic treatment, according to the embodiments herein. With respect to FIG. 7A-FIG. 7B, the colonies on the CM-coated dishes were found sensitive to these enzymes. The treatment with these enzymes caused colonies to collapse immediately. After the enzymatic treatment whole colonies immediately detached. The colonies cultured on the CM-coated plates were sensitive to enzymatic passaging. The scales of the images were kept 200 μm.

In addition the colonies were treated with another type of enzyme called as collagenase type IV with 1 mg/ml concentration (Invitrogen, 17104-019). It was found that the colonies on CM were resistant to collagenase type IV (1 mg/ml, Invitrogen, 17104-019) even after 10 minutes. The hESCs and hiPSCs were passaged on CM-coated plates mechanically. The colonies were cut in small pieces close to 0.2 mm² by keen edge of glass pipettes made on flame or STEMPRO® EZPassage™ tool (Invitrogen, 23181-010) followed by gently pipeting of colonies and replating on CM-coated plates. FIG. 7C shows an image of the colony cut by STEMPRO® EZPassage™ tool, according to an embodiment herein. With respect to FIG. 7C, the cut portion of the colonies can be seen. The scale is kept at 200 μm. FIG. 7D shows an image of the colony pieces ready to transfer to the new vessels, according to an embodiment herein. With respect to FIG. 7D, the pieces of the colonies are visible. The scale is kept at 200 μm. FIG. 7E shows an image of a part of the colony after two days of culturing on RoGel or CM, according to an embodiment herein. With respect to FIG. 7E, the scale is kept at 200 μm and the part of the colony is visible at this scale. FIG. 7F shows an image of a part of the colony after seven days of culturing on RoGel or CM, according to an embodiment herein. With respect to FIG. 7F, the scale is kept at 200 μm and the part of the colony after seven days is clearly visible.

Testing the Effect of Compatibility of RoGel:

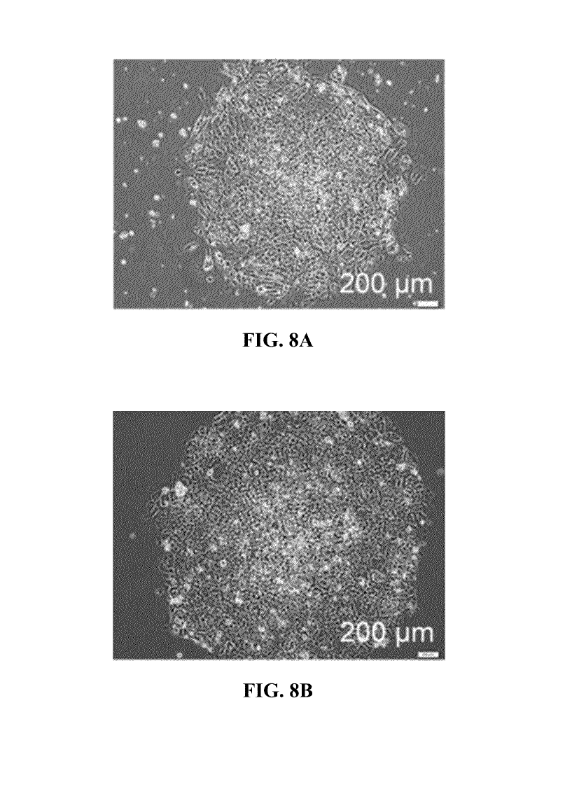

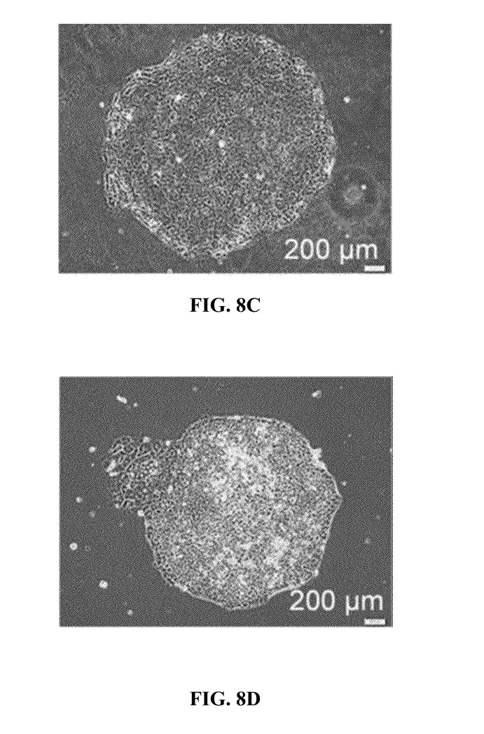

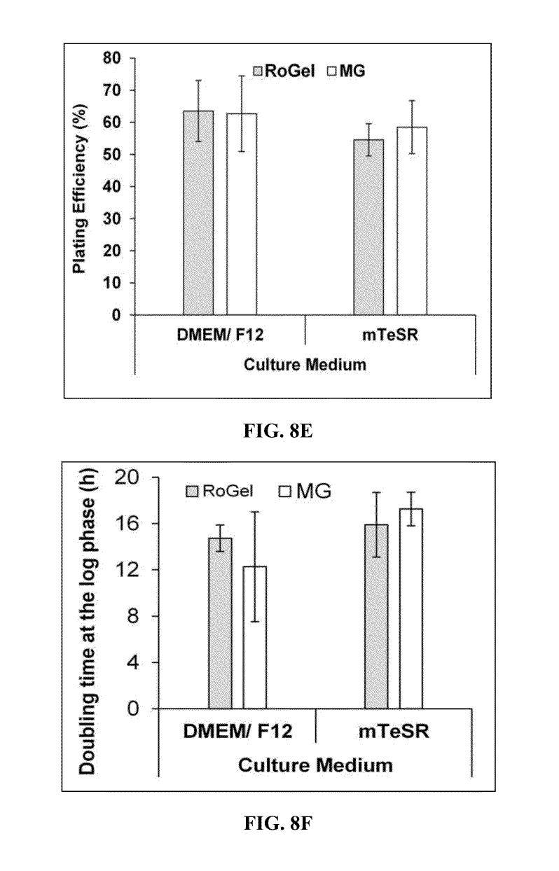

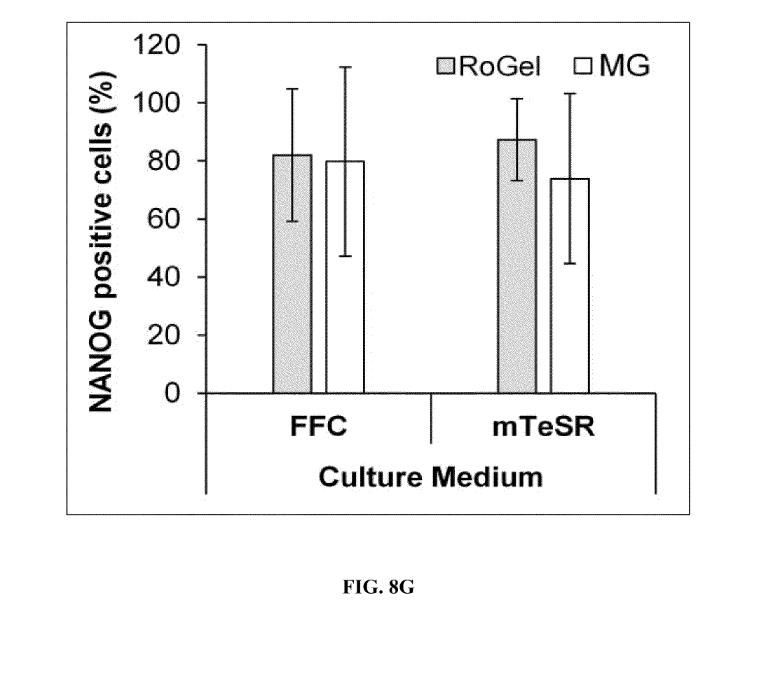

To evaluate the compatibility of “RoGel” or the CM with different culture systems, its ability to support self-renewal of Royan H6 hESCs in both commercially available and chemically defined medium mTeSR®1 (Stemcell Technologies, 05857), in comparison with the hESC medium-based DMEM/F12 as a control on MG and CM for 5-7 passages was tested. The Royan H6 hESCs were cultured for seven days. The hPSCs were maintained for at least five passages on “RoGel” in DMEM/F12 and the mTeSR1 media. FIG. 8A-FIG. 8D shows images of the grown colonies in presence of CM or the RoGel with other commercially available and chemically defined mediums, where FIG. 8A shows an image showing the growth of a colony when cultured on CM containing DMEM/F12 in addition with KOSR, where FIG. 8B shows an image showing the growth of a colony when cultured on CM in presence of mTeSR1, where FIG. 8C shows an image showing the growth of a colony on MG in presence of DMEM/F 12 and KOSR and where FIG. 8D shows an image showing the growth of a colony on MG in presence of mTeSR1, according to embodiments herein. With respect to FIG. 8A-FIG. 8D, growth of the hESCs were seen. The scale was kept at 200 μm.

FIG. 8E shows a histogram showing the effect of compatibility of CM with DMEM/F12 and mTeSR culture mediums, according to the embodiments herein. With respect to FIG. 8E, the cells showed similar plating efficiency (PE).

FIG. 8F shows a histogram showing the doubling time population in presence of CM with DMEM/F12 and mTeSR culture mediums, according to the embodiments herein. With respect to FIG. 8F, the cells showed similar doubling time.

FIG. 8G shows a histogram showing the expression of sternness-specific marker i.e. NANOG, according to the embodiments herein. With respect to FIG. 8G, the cells showed similar expression. The NANOG positive cells were found similar in presence of different types of culture mediums. The property of self-renewal of Royan H6 hESCs in both media on two different substrates was found efficient. The cells retained important hESC characteristics, such as doubling time and specific marker expression, NANOG as detected by flow cytometry.

Freeze/Thaw of hPSCs on “RoGel”:

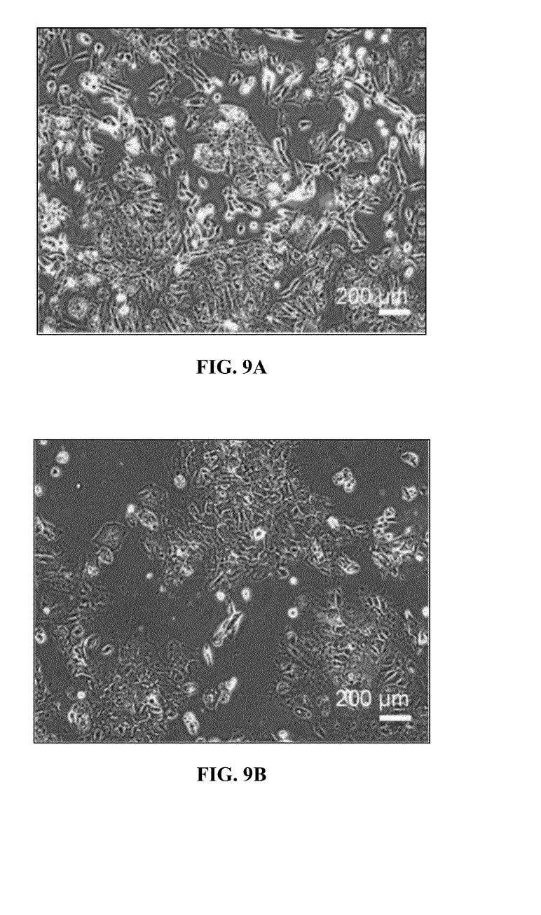

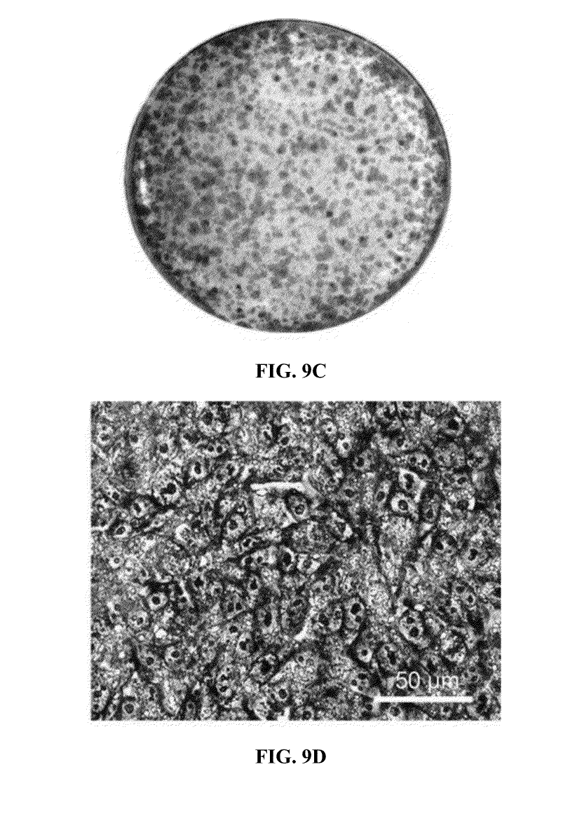

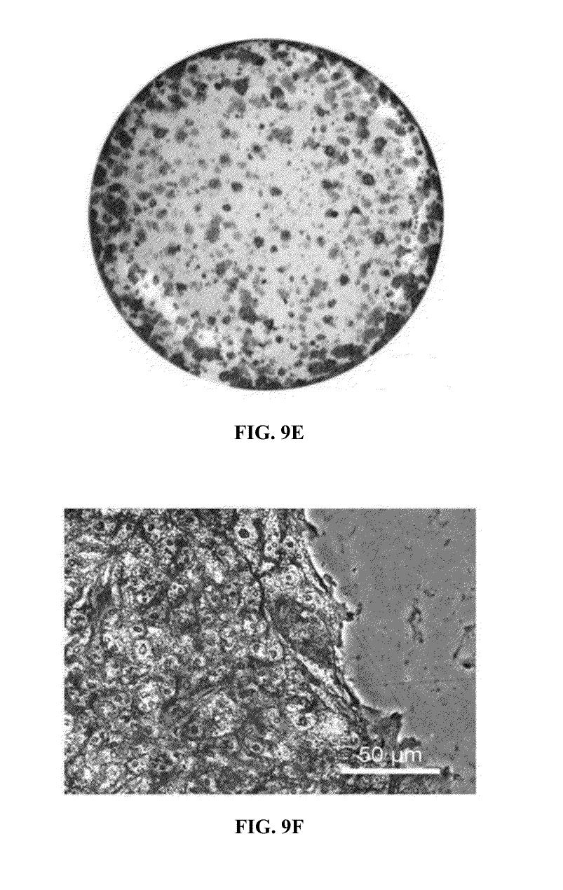

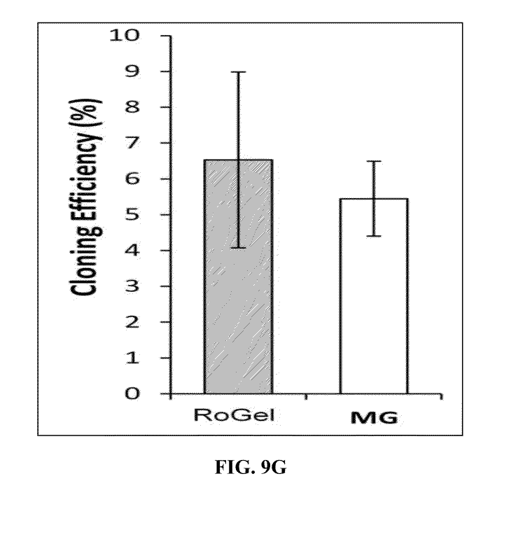

It was assessed whether the cells maintained on “RoGel” could be cryopreserved and then thawed back onto “RoGel” or not. For this experiment, the Royan H6 hESCs that had been maintained for nine passages were cryopreserved and then thawed back onto “RoGel”. FIG. 9A shows a photograph showing the phase contrast of hESCs i.e. Royan H6 grown on the CM, according to an embodiment herein. FIG. 9B shows a photograph showing the phase contrast of hESCs i.e. Royan H6 grown on MG, according to the embodiment herein. With respect to FIG. 9A and FIG. 9B, the phase contrast in both the mediums was found similar. The scale was kept at 200 μm.

FIG. 9C shows a photograph showing the hESCs or Royan H6 cell line by Alkaline Phosphatase (ALP) staining on the CM, according to an embodiment herein. With respect to FIG. 9C, the scale is 200 μm and the ALP positive cell lines are visible.

FIG. 9D shows a photograph showing the hESCs or Royan H6 cell line by Alkaline Phosphatase (ALP) staining on the CM at a larger scale, according to an embodiment herein. With respect to FIG. 9D, the scale is 50 μm and the ALP positive cell lines are more prominently visible.

FIG. 9E shows a photograph showing the hESCs or Royan H6 cell line by Alkaline Phosphatase (ALP) staining on the MG, according to an embodiment herein. With respect to FIG. 9E, the scale is 200 μm and the ALP positive cell lines are visible on MG substrate.

FIG. 9F shows a photograph showing the hESCs or Royan H6 cell line by Alkaline Phosphatase (ALP) staining on the MG at a larger scale, according to an embodiment herein. With respect to FIG. 9F, the scale is 50 μm and the ALP positive cell lines are more prominently visible.

FIG. 9G shows a histogram showing an evaluation of the cloning efficiency for cultured hPSCs, according to an embedment herein. The cloning efficiency was analyzed on the basis of the ratio of ALP-positive colonies formed per initially seeded hESC by ImageJ software. With respect to FIG. 9G, the recovery was similar to cells maintained on MG and thawed onto MG with the cells maintained on the CM and thawed on CM in terms of both cloning efficiency and ALP expression.

Characterization of Pluripotency Markers of hPSCs on “RoGel”:

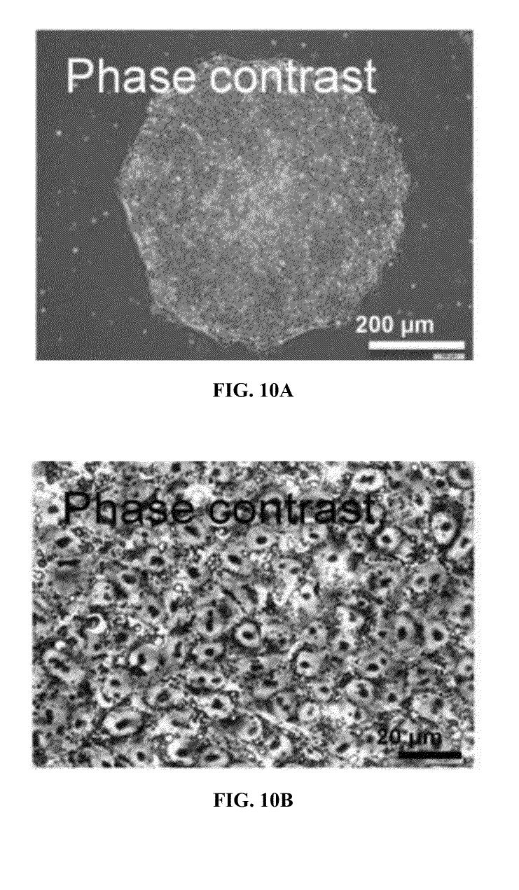

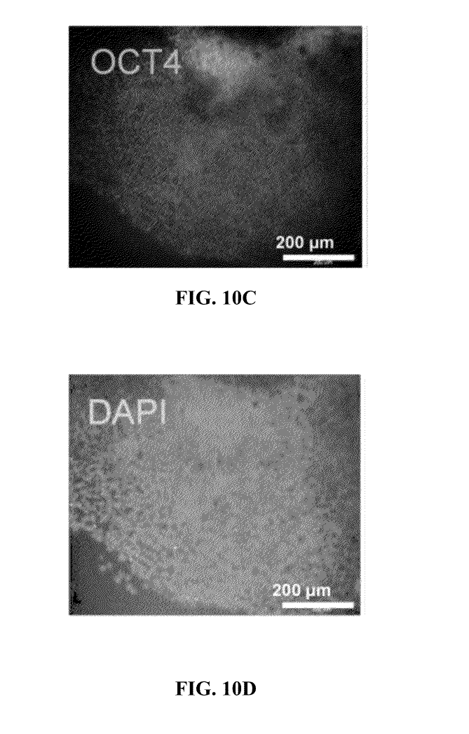

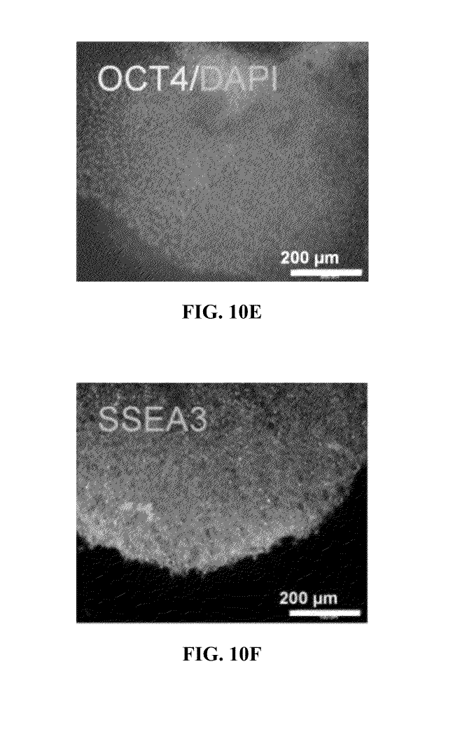

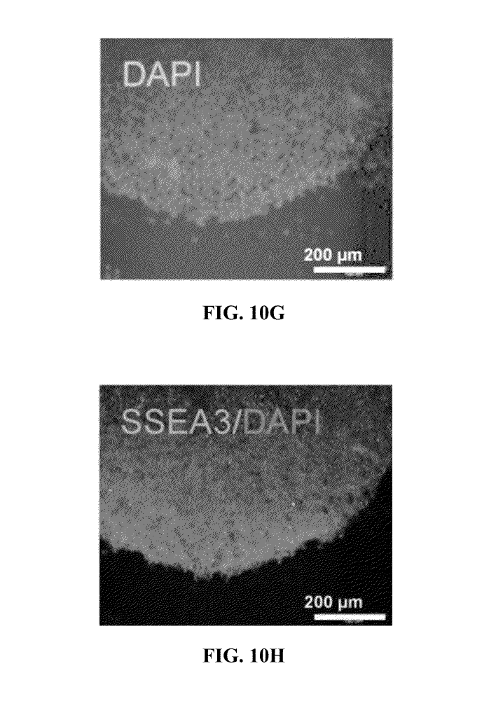

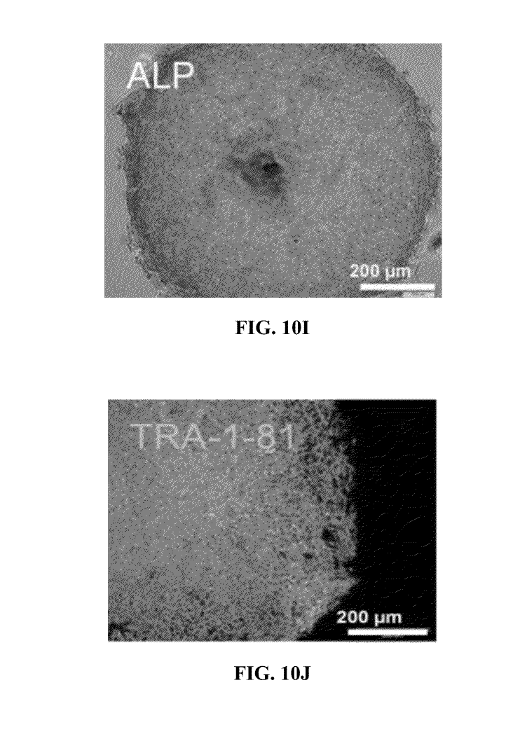

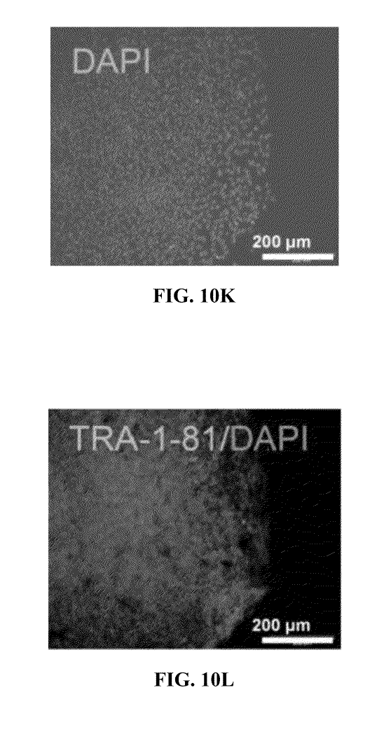

The pluripotency maintenance is a critical parameter when evaluating new ECM for hPSCs culture. The cells were cultured on “RoGel” for more than ten serial passages in hESC medium. The morphology of the colony and the expression of hPSC marker were evaluated at the end of each passage. The typical hPSC colony morphology for cells grown on CM was similar to those of MG cultures. FIG. 10A shows an image showing the phase contrast of hESCs, according to an embodiment herein. With respect to FIG. 10A, the scale was kept at 200 μm and the grown colony can be seen. FIG. 10B shows an image showing the phase contrast of hESCs at a magnified scale, according to an embodiment herein. With respect to FIG. 10B, the scale is kept at 20 μm and the cells lines are clearly visible.

Immuno-fluorescence evaluation of hPSC-specific markers, OCT4, SSEA3 and TRA-1-81 showed expressions of markers cultured on “RoGel” and MG control cultures. The hPSCs also expressed NANOG, SOX2 and KLF4 as detected by RT-PCR. The lines are characterized after five to twenty passages.

FIG. 10C shows an image showing the expression of hESCs in presence of OCT4 marker, according to the embodiments herein. With respect to FIG. 10C, the expression of hESCs in presence of OCT4 marker is visible. The scale is kept at 200 μm. FIG. 10D shows an image showing the stained nuclei with DAPI, according to the embodiments herein. With respect to FIG. 10D, the stained nuclei can be seen. The scale is kept at 200 μm. FIG. 10E shows an image showing the expression of hESCs in presence of OCT4 and DPAI, according to the embodiments herein. With respect to FIG. 10E, the scale is kept at 200 μm and the cell lines are visible. FIG. 10F shows an image showing the expression of hESCs in presence of SSEA3 marker, according to the embodiments herein. With respect to FIG. 10F, the scale is kept at 200 μm. FIG. 10G shows an image showing the stained nuclei with DAPI, according to the embodiments herein. With respect to FIG. 10G, the stained nuclei can be seen. The scale is kept at 200 μm. FIG. 10H shows an image showing the expression of hESCs in presence of SSEA3 marker and DPAI stain, according to the embodiments herein. With respect to FIG. 10H, the expression of hESCs in presence of SSEA3 and DPAI can be seen. The scale is kept at 200 μm. FIG. 10I shows an image showing the alkaline phosphatase staining of the hSECs, according to the embodiments herein. With respect to FIG. 10I, the alkaline phosphatase positive cells are visible. The scale is kept at 200 μm. FIG. 10J shows an image showing the expression of cells in presence of TRA-1-81 marker, according to the embodiments herein. With respect to FIG. 10J, the expression of cells in presence of TRE-1-81 marker can be seen. The scale is kept at 200 μm. FIG. 10K shows an image showing the stained nuclei with DAPI stain, according to the embodiments herein. With respect to FIG. 10K, the stained nuclei can be seen. The scale is kept at 200 μm. FIG. 10L shows an image showing the expression of hESCs in presence of TRA-1-81 marker and DPAI stain, according to the embodiments herein. With respect to FIG. 10L, the expression of hESCs in presence of TRA-1-81 marker and DPAI stain is visible. The scale is kept at 200 μm. The hPSCs on “RoGel” or CM retained key properties of pluripotent markers.

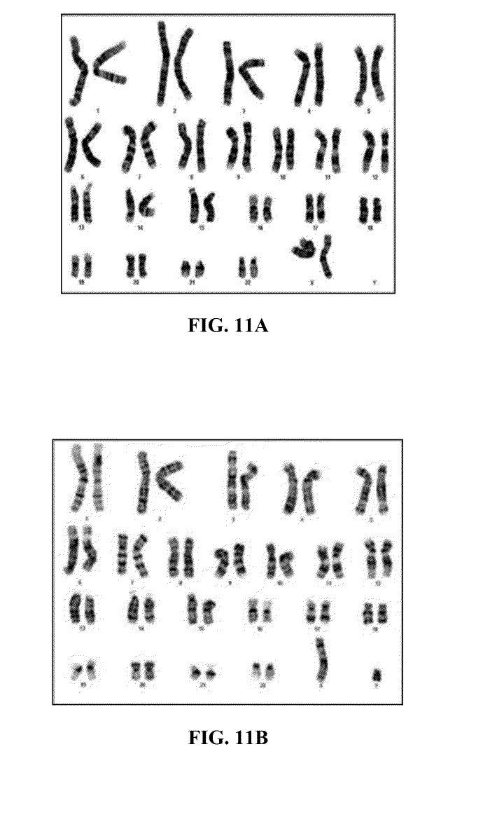

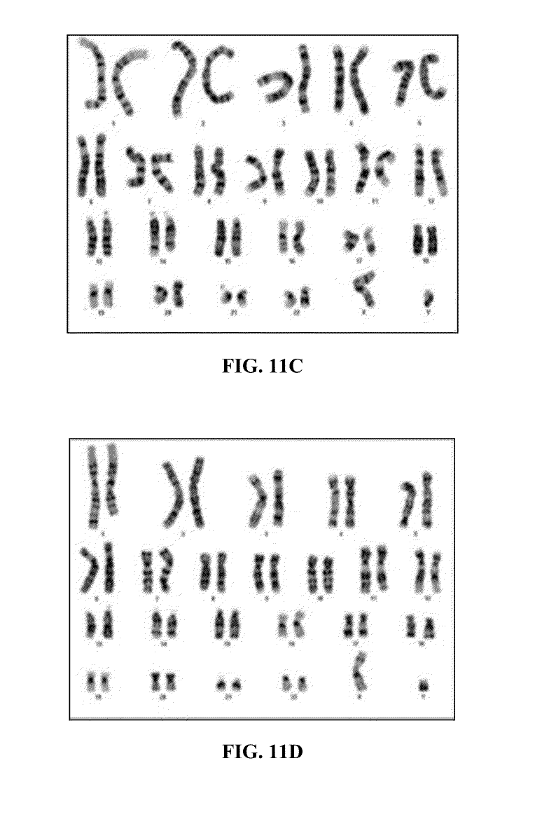

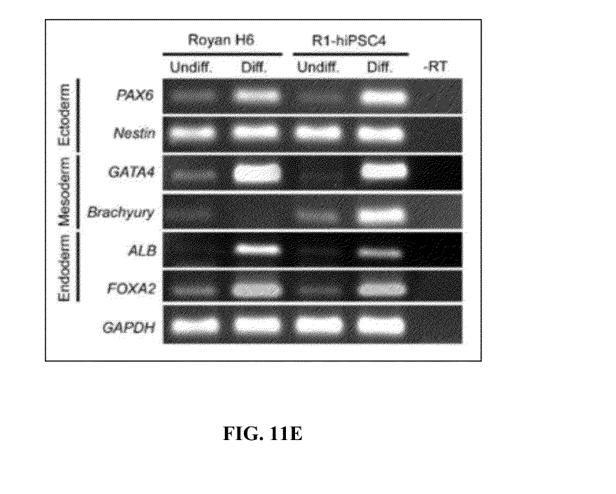

A karyotype analysis was performed. The hESCs and hiPSCs were propagated on “RoGel” to demonstrate the absence of chromosomal abnormalities. The results showed normal karyotype for both hESC and hiPSC lines. FIG. 11A shows a karyotype of Royan H5 cell line, according to the embodiments herein. With respect to FIG. 11A, the karyotype of Royan H5 cell line shown is of Passage no. 13. FIG. 11B shows karyotype of Royan H6 cell line, according to the embodiments herein. With respect to FIG. 11B, the karyotype of Royan H6 cell line shown is of Passage no. 14. FIG. 11C shows karyotype of R1-hiPSC1 cell line, according to the embodiments herein. With respect to FIG. 11C, the karyotype of R1-hiPSC1 cell line is of Passage no. 18. FIG. 11D shows karyotype of R1-hiPSC4 cell line, according to the embodiments herein. With respect to FIG. 11D, the karyotype of R1-hiPSC4 cell line is of Passage no. 11. With respect to FIG. 11A to FIG. 11D, the karyotype of hESCs and hiPSCs after several passages on “RoGel” was normal.

The pluripotency of hPSCs maintained on “RoGel” was also assessed by in vitro spontaneous differentiation of Embryoid Bodies (EBs). FIG. 11E shows RT-PCR analysis of in vitro differentiation of hESCs i.e. Royan H5 cell lines by EB formation showing the expression of the markers of the three embryonic germ layers, according to the embodiments herein. With respect to FIG. 11E, the tissue components that expressed markers of the three germ layers such as: FOXA2 and ALB for the endoderm, PAX6 and Nestin for the ectoderm, and Brachyury and GATA4 for the mesoderm were all detected by RT-PCR. According to the embodiments herein, the CM or “RoGel” has potential to be used for directed differentiation into hepatocytes from hPSCs comparable with MG.

Finally, the pluripotency of hPSC i.e. Royan H6 and R1-hiPSC4 cells was examined. The pluripotency was examined by the ability of the cells retained after 12 and 7 passages in hESC medium on CM-coated dishes to generate teratomas in nude mice. Twelve weeks after transplantation, teratomas had formed and histological analyses revealed the presence of cells from all three germ layers. FIG. 11F-FIG. 11I show images showing the formation of teratomas and further showing the presence of cells from all the three germ layers, where FIG. 11F shows a macroscopic view of the teratoma with distinct retinal pigment epithelium (RPE) on the teratoma, where FIG. 11G shows an image showing the microscopic view of the formed teratoma showing the Retinal Pigment Epithelium (RPE), where FIG. 11H shows an image showing the microscopic view of the formed teratoma showing the cartilage, where FIG. 11I shows an image showing the microscopic view of the formed teratoma showing the intestinal epithelium, according to the embodiments herein. With respect to FIG. 11G, the RPE is the ectodermal marker. With respect to FIG. 11H, the cartilage is the mesodermal marker and with respect to FIG. 11I, the intestinal epithelium is the endodermal marker. Thus with respect to FIG. 11F-FIG. 11I, the presence of cells of all the three germ layers can be seen.

Fractionation of “RoGel” to Support Self-Renewal of hPSCs:

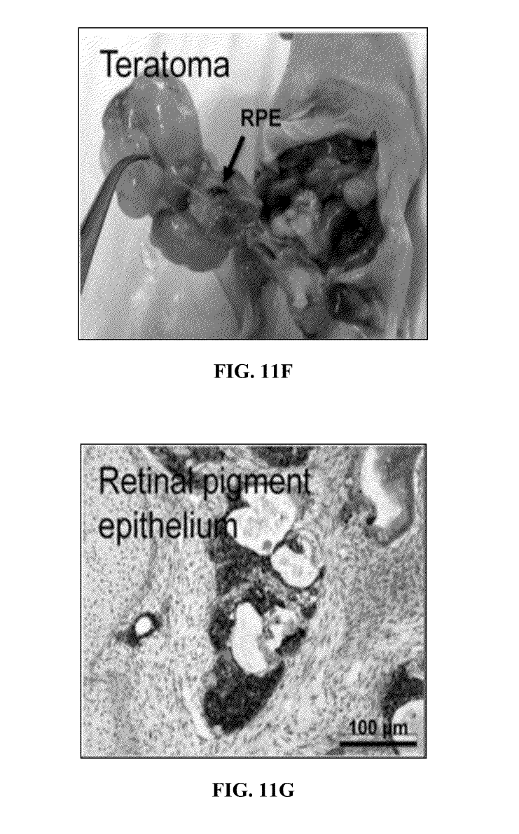

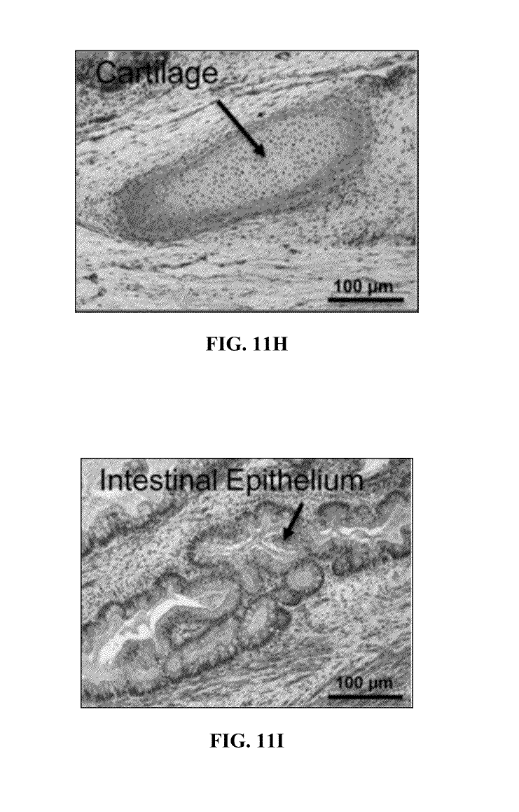

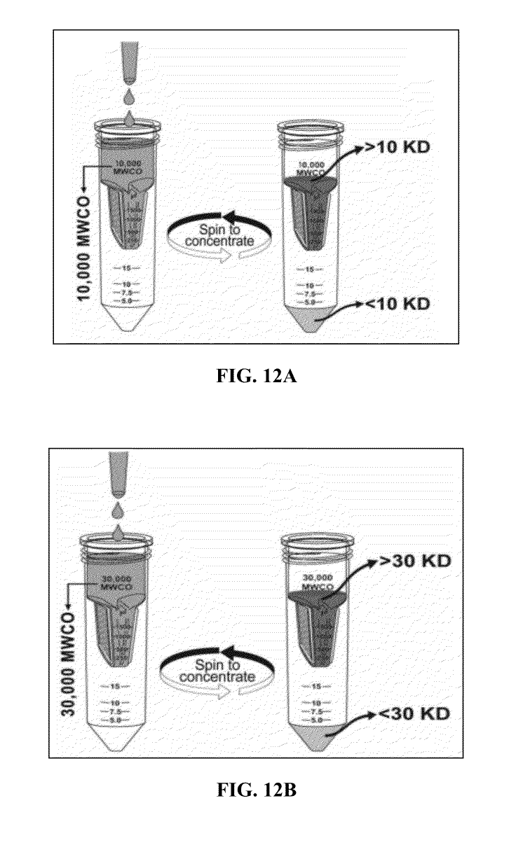

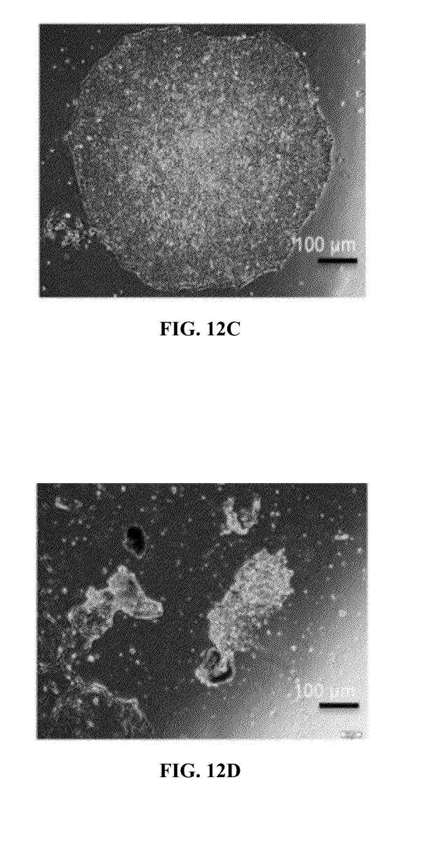

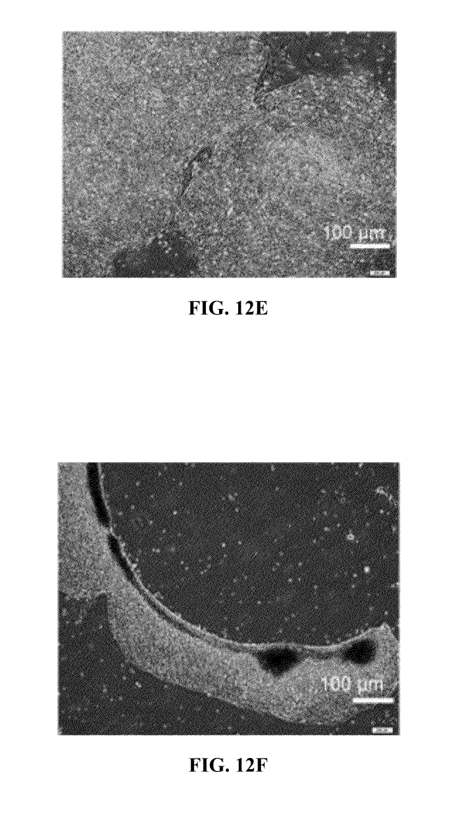

To assess the effect of fractionation of the CM as support on the self-renewal of the hPSCs, >10 KD, <10 KD, >30 KD and <30 KD fractionated “RoGel” was used. The effective fraction of CM in the undifferentiated growth of hESCs and hiPSCs was analyzed. The plates were separately coated by fractionated CM. The Royan H6 hESCs were cultured on the four fractions. FIG. 12A and FIG. 12B shows a diagrammatic representation of a Millipore's Amicon Ultra-15 centrifugal filter device showing the fractionation of the CM, where FIG. 12A shows a diagrammatic representation of a Millipore's Amicon Ultra-15 centrifugal filter device showing the fractionation of the CM to >10 KD and <10 KD based on the Molecular Weight of CM's proteins (MWCO) and where FIG. 12B shows a diagrammatic representation of a Millipore's Amicon Ultra-15 centrifugal filter device showing the fractionation of the CM to >30 KD and <30 KD based on the Molecular Weight of CM's proteins (MWCO), according to an embodiment herein. FIG. 12C-FIG. 12F show images of the colonies of the hPSCs grown on the fractionated CM, where FIG. 12C shows an image showing the growth of hPSCs on the >10 KD fractionated CM, where FIG. 12D shows an image showing the growth of hPSCs on the <10 KD fractionated CM, where FIG. 12E shows an image showing the growth of hPSCs on the >30 KD fractionated CM and where FIG. 12F shows an image showing the growth of hPSCs on the <30 KD fractionated CM, according to the embodiments herein. With respect to FIG. 12A-FIG. 12F, by analyzing the morphology of the colony in the figures, it was found that the cells grew with hESC morphology when the plates were coated with >10 KD and >30 KD fractionated “RoGel” or CM. It was also found that the <10 KD CM and <30 KD CM that contained 10 KD structures were not suitable. Thus the structures with >30 KD were more important. The scale in the images was kept at 100 μm.

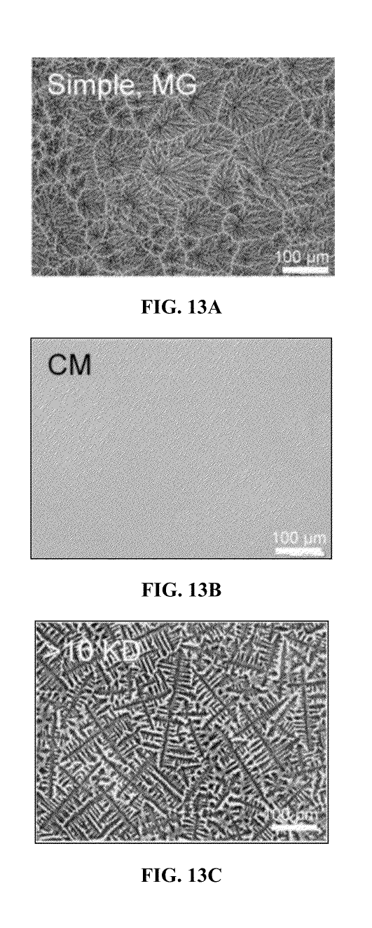

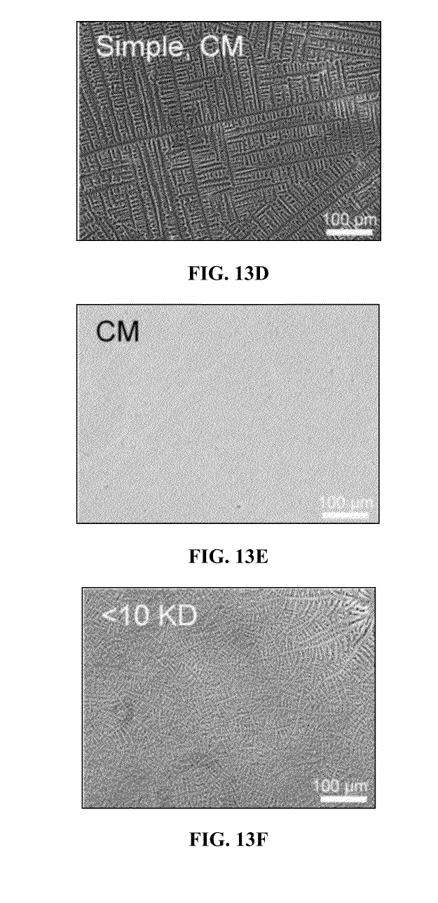

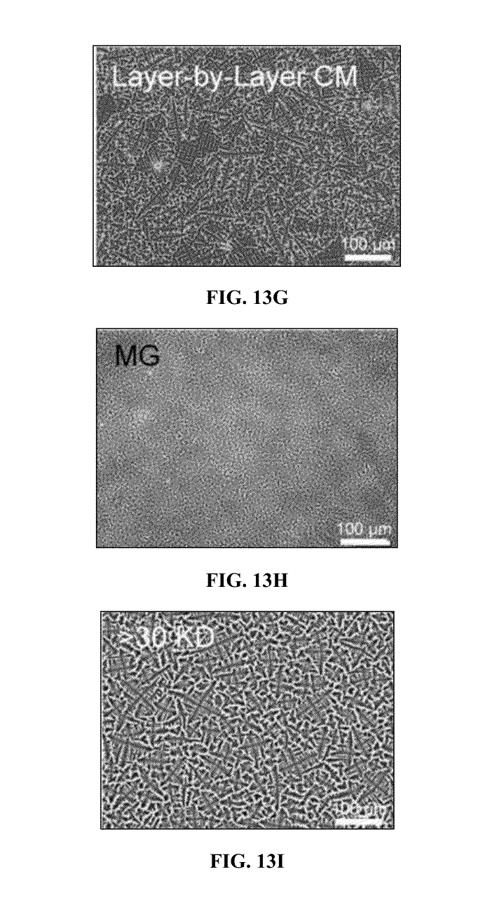

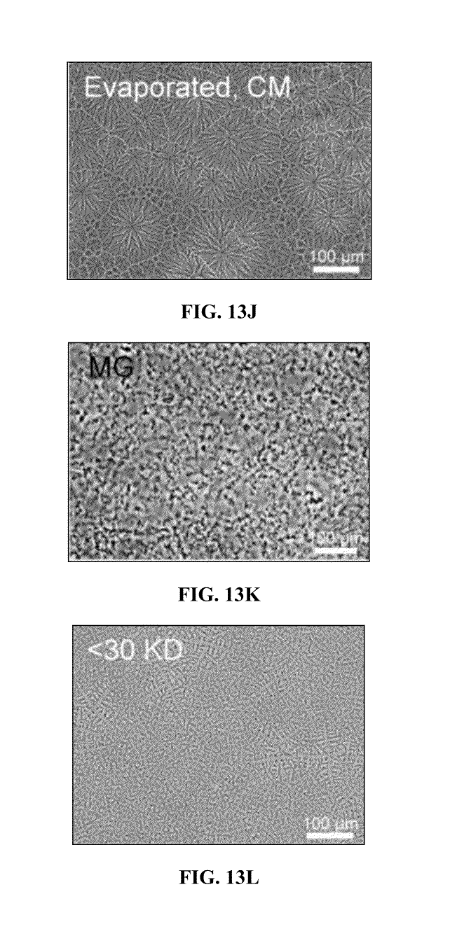

The shapes of coated surfaces by MG, simple or layer-by-layer RoGel or CM and fractionated RoGel or CM were focused on. FIG. 13A shows a phase contrast microscopy image of plate coated with simple layer of MG, according to the embodiments herein. With respect to FIG. 13A, the scale is 100 μm and the MG is air dried. The MG is a whole ECM. FIG. 13B shows a phase contrast microscopy image of plate coated with simple layer of Fresh CM, according to the embodiments herein. With respect to FIG. 13B, the scale is 100 μm. The CM used is fresh. FIG. 13C shows a phase contrast microscopy image of plate coated with >10 KD fractionated CM, according to the embodiments herein. With respect to FIG. 13C, the scale is 100 μm. FIG. 13D shows a phase contrast microscopy image of plate coated with simple layer of CM, according to the embodiments herein. With respect to FIG. 13D, the scale is 100 μm and the CM is air dried. The CM is whole ECM. FIG. 13E shows a phase contrast microscopy image of plate coated with simple layer of Fresh CM, according to the embodiments herein. With respect to FIG. 13E, the scale is 100 μm. The CM used is fresh. FIG. 13F shows a phase contrast microscopy image of plate coated with <10 KD fractionated CM, according to the embodiments herein. With respect to FIG. 13F, the scale is 100 μm. FIG. 13G shows a phase contrast microscopy image of plate coated with CM using layer-by-layer method, according to the embodiments herein. With respect to FIG. 13G, the scale is 100 μm and the CM is air dried. The CM is whole ECM. FIG. 13H shows a phase contrast microscopy image of plate coated with MG using layer-by-layer method, according to the embodiments herein. With respect to FIG. 13H, the scale is 100 μm and the MG used is a fresh MG. FIG. 13I shows a phase contrast microscopy image of plate coated with >30 KD fractionated CM, according to the embodiments herein. With respect to FIG. 13I, the scale is 100 μm. FIG. 13J shows a phase contrast microscopy image of plate coated with CM, according to the embodiments herein. With respect to FIG. 13J, the scale is 100 μm and the CM is evaporated. The CM is whole ECM. FIG. 13K shows a phase contrast microscopy image of plate coated with evaporated MG, according to the embodiments herein. With respect to FIG. 13K, the scale is 100 μm and the MG is fresh MG. FIG. 13L shows a phase contrast microscopy image of plate coated with <30 KD fractionated CM, according to the embodiments herein. With respect to FIG. 13I, the scale is 100 μm. According to FIG. 13A-FIG. 13I, it was found that the air dried-coated surfaces of “RoGel” or CM with >10 KD and >30 KD fractions were similar to air dried MG and whole “RoGel” or CM.

According to the embodiments herein, the human feeder-derived CM for self-renewal of hESCs and hiPSCs was successfully developed and applied. A successful maintenance of hPSCs on “RoGel” over long-term serial passages in a xeno-free and defined medium was observed. Reubinoff et al. described the impact of neurobasal medium on the maintenance of hPSCs on suspension culture. Other research groups then applied this medium for undifferentiating maintenance of hPSCs in large scale suspension culture systems as effective basal medium. According to the embodiments herein, a conditioned medium comprising the neurobasal medium without any serum or KOSR was prepared.

According to the embodiments herein, the hPSCs cultured on the conditioned medium maintained stable, typical hESC morphology, proliferation, expression of stem cell markers, in vitro pluripotency and normal karyotype. The results have demonstrated that the conditioned medium is sufficient to support long-term self-renewal of hESCs in a defined medium. This approach offers a simple, less intensive, less time consuming, in-house prepared and low-cost ECM for prolonged expansion of hESCs and hiPSCs with karyotype stability. The human feeder-derived CM decreases the risk of cellular contamination and animal-derived pathogens. The human feeder derived CM also provides a scalable, robust platform for the clinical grade culture of hPSCs. The HDF and HFF-derived CMs and even high passage i.e. passage 20 feeder-derived CMs have a similar effect on hPSC expansion. It is shown that long-term culture of feeders does not influence the quality of CM in support of hPSC self-renewal.

The research has also shown that expansion of commercially available chemically defined media such as mTeSR and KOSR-supplemented medium is suitable for hESC culture on the conditioned medium or “RoGel”. In addition, it is demonstrated that hPSCs cultured on the conditioned medium or the “RoGel” could be cryopreserved and successfully thawed back onto the “RoGel” for further expansion or differentiation, which is important for the production of clinical grade hPSC banks. Notably, it is demonstrated that ECM provides a scalable, enzyme-free passage of hPSCs that is important to maintain a stable cell karyotype. Finally, the hPSCs maintained a self-renewal on plates preserved for a long term i.e. one year, at room temperature. This property is important for the distribution of coated plates and the expansion of scalable hPSCs. The hPSCs grew when the plates were coated with >30 KD fractionated “RoGel”. Thus the secreted ECM molecules were important in secretion and growth factors with low molecular weight (<30 KD) were not important in this regard.

The ECM molecules are large, complex and often multimeric structures. Their recombinant production is generally difficult or not cost effective. Meanwhile, ECM is a natural mixture of molecules, hence CM mimics natural ECM. Several research groups have been revealed the proteome of CM of mouse and human fibroblast feeders in order to replace its critical molecules to establish a defined condition culture system for hPSCs. Recently it has been demonstrated that HDF secretes laminin-511 and expresses the laminin-binding integrins α3β1, α6β1 and α7β1. They have a critical role in the maintenance of hPSCs. Other efficient ECM molecules such as vitronectin and the recently reported E-cadherin type I and have a molecular weight of >30 KD.

In summary, a simple, robust, repeatable, in-house prepared, cost-effective ECM for the maintenance of hPSC self-renewal in a defined medium is developed. The ECM in the embodiments herein has the potential for large-scale expansion of hPSCs and directed differentiation in place of MG. We believe that this ECM will be useful for both research purposes and future therapeutic applications.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. Axeno-free and a feeder free self-renewal extracellular matrix for long-term maintenance of undifferentiated Human Embryonic Stem Cells (hESCs) and undifferentiated Human Pluripotent Stem Cells (hPSCs) comprising:
a conditioned medium, wherein the conditioned medium comprises a basal medium, wherein the basal medium is neurobasal medium;
a nutrition providing medium, wherein the nutrition providing medium is Dulbecco's Modified Eagle Medium/F12 (DMEM/F12);
a plurality of additives; and
a plurality of feeder cells, wherein the plurality of feeder cells are Human Dermal Fibroblasts (HDFs)inactivated by mitomycin C for 2 hours.

2. The extracellular matrix according to claim 1, wherein the plurality of additives areselected from a group consisting of L-glutamine (L-Gln), β-mercaptoethanol (β-ME), nonessential amino acids (NEAAs) and insulin-transferrin-selenite (ITS).

3. The extracellular matrix according to claim 2, wherein the nonessential amino acids (NEAAs) are selected from a group consisting of alanine, asparagines, aspartic acid, cysteine, cyctine, glutamine, glutathione, glycine, histidine, proline, serine, taurine and threonine.

4. The extracellular matrix according to claim 1, wherein the conditioned medium is added with a Rho-associated coiled kinase (ROCK) inhibitor Y-27632.

5. The extracellular matrix according to claim 1, wherein the conditioned medium is derived from human feeder cells, wherein the human feeder cells are Human Dermal Fibroblasts (HDFs).

6. The extracellular matrix according to claim 1, wherein the Human Dermal Fibroblasts are present in a high density value and wherein the Human Dermal Fibroblasts are present in a low density value, wherein the high density value is 50000 cell/cm² and wherein the low density value is 5000 cell/cm².

7. The extracellular matrix according to claim 1, wherein the conditioned medium maintains an undifferentiated Human Embryonic Stem Cells (hESCs) and an Human Pluripotent Stem Cells (hPSCs) for a period of one year at a room temperature.

8. The extracellular matrix according to claim 1, wherein the extracellular matrix is in the form of a gel.

9. A method of synthesizing a xeno-free and a feeder free self-renewal extracellular matrix for long-term maintenance of undifferentiated Human Embryonic Stem Cells (hESCs) and Human Pluripotent Stem Cells (hPSCs), the method comprises:
preparing a conditioned medium, wherein the conditioned medium is prepared by adding a basal medium, a nutrition providing medium, a plurality of additives and a plurality of feeder cells together, and wherein the basal medium is neurobasal medium, and wherein the nutrition providing medium is Dulbecco's Modified Eagle Medium/F12 (DMEM/F12), and wherein the plurality of feeder cells are Human Dermal Fibroblasts (HDFs) inactivated by mitomycin C for 2 hours; and incubating the conditioned medium for a pre-determined amount of time at a pre-determined temperature.

10. The method according to claim 9, wherein the pre-determined amount of time is 24 hours-72 hours.

11. The method according to claim 9, wherein the pre-determined amount of temperature is 37° C.

12. The method according to claim 9, wherein the plurality of additives includes L-glutamine (L-Gln), β-mercaptoethanol (β-ME), nonessential amino acids (NEAAs), and insulin-transferrin-selenite (ITS).

13. The method according to claim 12, wherein the nonessential amino acids (NEAAs) are alanine, asparagines, aspartic acid, cysteine, cyctine, glutamine, glutathione, glycine, histidine, proline, serine, taurine and threonine.

14. The method according to claim 9, wherein the extracellular matrix maintains an undifferentiated Human Embryonic Stem Cells (hESCs) and an Human Pluripotent Stem Cells (hPSCs) for a period of one year at a room temperature.

15. The method according to claim 9, wherein the extracellular matrix is in the form of a gel.

16. The method according to claim 9 further comprises:
preparing a culture plate for culturing a Human Embryonic Stem Cells (hESCs) and a Human Pluripotent Stem Cells (hPSCs), wherein the step of preparing the culture plates comprises:
coating a plate with a conditioned medium for a predetermined amount of time, wherein the coating is simple coating and wherein the coating is layer-by-layer coating, and wherein the predetermined amount of time is 5 min-15 min.

17. The method according to claim 16, wherein the simple coating comprises:
adding the conditioned medium on a plate;
swirling the plate to evenly spread the conditioned medium;
incubating the plate at a predetermined temperature for the predetermined amount of time, wherein the pre-determined temperature is 37° C.;
drying the plate, wherein the plate is dried at a room temperature for 15-20 min.

18. The method according to claim 16, wherein the layer-by-layer coating comprises:
adding the conditioned medium on a plate;
swirling the plate to evenly spread the conditioned medium;
incubating the plate at a predetermined temperature for the predetermined amount of time, wherein the pre-determined temperature is 37° C.;
drying the plate, wherein the plate is dried at room temperature for 15-20 min; and
recoating the plate for a plurality of times.

19. The method according to claim 16, wherein the conditioned medium is added with a Rho-associated coiled kinase (ROCK) inhibitor Y-27632.

20. The method according to claim 16, wherein the plates are coated with fractionated conditioned medium with a range of 10 KD-30 KD.


SEQUENCE LISTING (14 sequences)

SEQ ID NO: 1 - protein, 4 aa, Artificial Sequence
  Synthetic Peptide
GHRP

SEQ ID NO: 2 - protein, 4 aa, Artificial Sequence
  Synthetic Peptide
KHRR

SEQ ID NO: 3 - protein, 4 aa, Artificial Sequence
  Synthetic Peptide
AAAA

SEQ ID NO: 4 - protein, 7 aa, Artificial Sequence
  Synthetic Peptide
GHRPSYQ

SEQ ID NO: 5 - protein, 7 aa, Artificial Sequence
  Synthetic Peptide
GHRPSYQ

SEQ ID NO: 6 - protein, 6 aa, Artificial Sequence
  Synthetic Peptide
GDRPSY

SEQ ID NO: 7 - protein, 4 aa, Artificial Sequence
  Syenthetic peptide
PSYQ

SEQ ID NO: 8 - DNA, 35 bp, Artificial Sequence
  Synthetic primer
cggcctatgacctctagctcctcttctgaatcggg

SEQ ID NO: 9 - DNA, 31 bp, Artificial Sequence
  Synthetic primer
ggtcataggccgtcatatcaaggaaacagtg

SEQ ID NO: 10 - DNA, 18 bp, Artificial Sequence
  Synthetic primer
gatctgccaccatggatg

SEQ ID NO: 11 - DNA, 25 bp, Artificial Sequence
  Synthetic primer
tggtctagatcacggtcgcatgttg

SEQ ID NO: 12 - DNA, 29 bp, Artificial Sequence
  Synthetic primer
agcgaagattgcagcctgccaggggtggg

SEQ ID NO: 13 - DNA, 41 bp, Artificial Sequence
  Synthetic primer
caatcttcgctgctgcagctgcctcgccaggagaaaggttc

SEQ ID NO: 14 - DNA, 24 bp, Artificial Sequence
  Synthetic primer
agatctgccaccatggatgcaatg